(19) United States
(12) Patent Application Publication
(10) Pub. No.: US 2026/0294792 A1
(43) Pub. Date: Oct. 1, 2026

(54) DISSOLVABLE MICRONEEDLE DELIVERY SYSTEMS FOR BIOACTIVE COMPOUNDS

(71) Applicant: Yubeck, Inc., Des Plaines, IL (US)

(72) Inventors: Chris Baek, Des Plaines, IL (US); Matthew Gdovin, Spring Branch, TX (US); Kevin Ita, American Canyon, CA (US)

(73) Assignee: Yubeck, Inc., Des Plaines, IL (US)

(21) Appl. No.: 19/093,256

(22) Filed: Mar. 28, 2025

(51) Int. Cl. A61K 9/00; A61K 36/02; A61K 38/16

(52) U.S. Cl. CPC A61K 9/0021; A61K 36/02; A61K 38/164

(57) ABSTRACT

A dissolvable microneedle (DMN) system is provided for the transdermal, intradermal, topical, or transmucosal delivery of bioactive compounds. Each microneedle is formed from a biocompatible, biodegradable material that dissolves upon contact with interstitial or mucosal fluid, enabling localized or systemic administration. The system supports a range of bioactive agents, including vitamins, minerals, peptides, proteins, amino acids, antioxidants, algae-derived compounds, plant-derived volatile compounds, and synthetic molecules, including those encapsulated in nanocarriers such as lipid nanoparticles, liposomes, or polymeric systems. Microneedle arrays may be fabricated in customizable sizes and geometries, including layered or tip-loaded structures. The invention addresses challenges related to poor bioavailability, compound instability, or patient compliance associated with oral or injectable routes. Applications include therapeutic, cosmetic, veterinary, nutraceutical, and agricultural use. The system enables controlled, sustained, or biphasic release of sensitive compounds and expands delivery to populations with swallowing difficulties, malabsorption, or needle aversion, offering a versatile, minimally invasive platform for precision bioactive administration.

### BACKGROUND

### Field of the Invention

The present invention relates to the field of bioactive compound delivery systems, more specifically, to dissolvable microneedle (DMN) based systems designed for localized or systemic administration of biologically active agents. The invention includes biodegradable microneedle arrays that dissolve upon application to the skin or mucosal tissue, enabling the topical, transdermal, intradermal, or transmucosal delivery. The DMN system supports the delivery of a wide range of compounds including vitamins, minerals, proteins, peptides, amino acids, algae-derived materials, antioxidants, botanical extracts, plant-derived volatile compounds, synthetic small molecules, and encapsulated agents for use in pharmaceutical, nutraceutical, cosmetic, veterinary, and agricultural applications.

### Background of the Invention

Biologically active compounds such as proteins, peptides, amino acids, vitamins, minerals, and antioxidants present unique delivery challenges. Oral administration may result in low bioavailability due to degradation in the gastrointestinal tract, variable absorption, and first-pass hepatic metabolism. Parenteral routes, while effective, are invasive, often painful, and may require clinical oversight. Passive transdermal patches are generally limited to small, lipophilic molecules due to the stratum corneum barrier, limiting their use for larger or hydrophilic compounds.

Dissolvable microneedle (DMN) systems offer an emerging alternative by enabling minimally invasive delivery of active compounds through microstructures that penetrate the skin or mucosal tissue and dissolve upon contact with interstitial or mucosal fluid. The DMN system allows for both local and systemic delivery and may be adapted to support immediate, sustained, or biphasic release of active ingredients. There remains a need for delivery platforms capable of supporting a broader range of bioactive compounds such as those that are unstable, temperature sensitive, oxidation-prone, poorly soluble, or of high molecular weight across pharmaceutical, cosmetic, nutraceutical, veterinary, and agricultural applications.

The present invention addresses this need by providing a DMN system compatible with a diverse array of active ingredients, including water and fat soluble vitamins, essential and trace minerals, proteins, peptides, amino acids (including algae-derived and bioengineered forms), polyphenols, synthetic small molecules, and complex compounds requiring stabilization or controlled release. The system is capable of incorporating advanced carrier systems, such as solid lipid nanoparticles (SLNs), nanostructured lipid carriers (NLCs), liposomes, polymeric nanoparticles (e.g., PLGA, PEGylated), micelles, nanocrystals, cyclodextrins, and hybrid or depot-forming systems. Additional carriers include niosomes, ethosomes, nanocrystals, protein-based nanoparticles, mesoporous silica nanoparticles, exosomes, cubosomes, and biopolymer-based carriers.

Delivery routes supported by the invention include transdermal, intradermal, topical, transmucosal (e.g., buccal, gingival, or sublingual), and veterinary routes (e.g., ear, gumline, or flank). Additionally, the invention encompasses agricultural applications, including delivery of bioactives to plants through foliar, root-zone, or biodegradable hydrogel-embedded formats for crop support, stress resistance, and micronutrient replenishment.

The microneedle array may be adapted for diverse anatomical targets using customizable shapes (e.g., conical, pyramidal, barbed), dimensions (100-2,000 μm height), densities, and delivery surfaces (e.g., circular patches, gumline strips, full-face masks, wearable fabrics and wraps). Indications addressed may include nutrient deficiencies, metabolic or inflammatory disorders, skin and scalp care, neurodegenerative diseases, wound healing, stress recovery, contraception, and veterinary or plant-based applications.

This invention provides a scalable, stable, and biocompatible DMN delivery platform with broad applicability across pharmaceutical, cosmetic, nutraceutical, veterinary, and agricultural applications. It supports the minimally invasive administration of bioactive compounds that may be challenging to deliver using conventional methods.

### SUMMARY OF THE INVENTION

An object of the present invention is to provide a transdermal drug delivery system that can deliver larger bioactive molecules through the skin.

Another object of the present invention is to provide a transdermal drug delivery system that can deliver substances through the skin more quickly than a standard patch.

Another object of the present invention is to provide a transdermal drug delivery system that can deliver algae-based, Spirulina-based, or C-Phycocyanin-based nanoparticles through the skin over a sustained period of time.

The present invention provides DMN systems that deliver bioactive compounds through the skin for localized or systemic effect. The DMN system improves bioavailability and patient compliance compared to oral or injectable routes by offering a painless and self-administered platform that can be customized for a wide variety of therapeutic, cosmetic, and wellness applications.

Another object of the present invention is to provide a drug delivery system that improves patient compliance.

In an aspect of the invention, a dissolvable microneedle system is provided, comprising an array of dissolvable microneedles, each microneedle capable of penetrating a living being's skin. Each microneedle comprises a biocompatible material that is capable of dissolving in a living being's interstitial fluid, and at least one bioactive compound. The bioactive compound could comprise proteins, peptides, amino acids, antioxidants, polysaccharides, polyphenols, pigments, fatty acids, vitamins, minerals, or combinations thereof. In an embodiment, the bioactive compound is derived from algae. The bioactive compound could be Spirulina or C-Phycocyanin. In an embodiment, the bioactive compound is embedded in solid lipid nanoparticles.

In an embodiment, the bioactive compound is a stabilized biologic, encapsulated in a carrier.

In an embodiment, the biocompatible material could be hyaluronic acid, pullulan, chitosan, or alginate; they could also be cross-linked. Each microneedle could also comprise an excipient, a nanoparticle-stabiizing agent, or both.

In an embodiment, the microneedles could have two or more parts, wherein one part dissolves faster than the other. The microneedles could be configured for immediate, biphasic, sustained, or controlled release.

The system could be configured as a face mask, spot patch, gumline strip, full-body wrap, under-eye crescent, lip patch, oral insert, transdermal wearable, veterinary patch, textile item, or agricultural film for application to plant surfaces.

In an embodiment, the microneedles could be pyramidal, conical, or tapered. The microneedles could have a height to base ratio between 1.5:1 and 5:1. The microneedles could have height ranging from 100 μm to 2000 μm, or a base ranging from 100 μm to 500 μmin diameter. The spacing between neighboring microneedles could range from 300 μm to 1200 μm. Each microneedle could have a minimum fracture force that is greater than 0.058N and an insertion force ranging from 0.1 N to 3.0 N.

In an embodiment, each microneedle could have two parts, wherein one part dissolves faster than the second part.

In an embodiment, the system could also comprise an embedded indicator configured to indicate whether or not the bioactive compound has been absorbed or the amount of compound remaining. The indicator could be visual, chemical, or any other indicator.

Methods of treating various conditions by means of the DMN system are also disclosed. The conditions could be neurological or psychological, digestive, nutritional deficiencies, autoimmune disorders, post-viral or infectious diseases. The bioactive compound could also be configured to modulate inflammation, oxidative stress, sleep, athletic performance, immune function, neurological activity, hormonal balance, or metabolic processes.

In an embodiment, the bioactive compound could be configured to support hair growth, manage scalp inflammation, manage sebum balance, manage the skin microbiome, support skin regeneration, hydration, wound healing, melanin, skin aging, or collagen production.

### LIST OF FIGURES

FIG. 1 shows a diagram of the operation of an embodiment of the present invention.

FIG. 2 shows a diagram of the process of manufacturing an embodiment of the present invention.

### DETAILED DESCRIPTION

### Overview

Dissolvable microneedles are arrays of microscopic needles composed of biodegradable, water-soluble materials. When applied to the skin, the microneedles painlessly pierce the stratum corneum, creating microchannels through which encapsulated nutrients are delivered into the dermis. The microneedles then dissolve, releasing their payload directly into the interstitial fluid for systemic absorption. DMNs offer several advantages over traditional transdermal patches, including their ability to bypass the passive diffusion mechanism, deliver larger molecules (>500 Da), and provide sustained release of active ingredients. For example, DMNs could be used to deliver large molecules such as phycocyanin encapsulated in a solid lipid nanoparticle (SLN) transdermally, which would be impossible in a standard transdermal system. Standard transdermal patches rely on passive diffusion through the stratum corneum making them unsuitable for delivering larger compounds that are >500 Daltons.

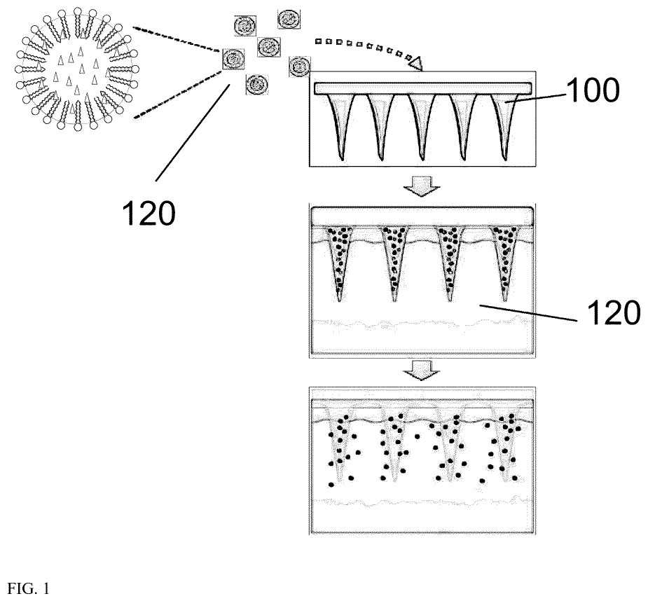

FIG. 1 shows a diagram of a DMN system being used to deliver solid lipid nanoparticle (SLNs) topically or transdermally. SLNs 110 are embedded in a plurality of microneedles 100. When the microneedles 100 are inserted into the skin 120, they dissolve over time and deliver SLNs 110 into the interstitial fluid. The structure and manufacture of SLNs is disclosed in App. No. Ser. No. 19/093,251.

### Composition

The present invention relates to a dissolvable microneedle-based delivery system comprising bioactive compounds such as vitamins, minerals, enzymes, biologics, algae-derived materials, proteins, peptides, amino acids, antioxidants, synthetic small molecules, and naturally derived phytochemical compounds such as botanical extracts and polyphenols.

As used herein, the term ‘algae’ includes microalgae, macroalgae, cyanobacteria (e.g., Spirulina), and other marine organisms capable of producing biologically active compounds.

In certain embodiments, the dissolvable microneedle (DMN) system may be adapted for the delivery of macromolecular biologics, including but not limited to monoclonal antibodies, cytokine inhibitors, fusion proteins, antibody fragments, aptamers, and recombinant protein therapeutics. These biologics may be employed in the treatment of autoimmune and chronic inflammatory conditions, including but not limited to Crohn's disease, ulcerative colitis, psoriasis, systemic lupus erythematosus (SLE), rheumatoid arthritis, and other immune-mediated diseases.

Representative biologics contemplated within the scope of this invention, such as IL-23 inhibitors (e.g., ustekinumab, risankizumab, guselkumab, tildrakizumab); IL-17 inhibitors (e.g., secukinumab, ixekizumab); TNF-α inhibitors (e.g., adalimumab, infliximab, etanercept); IL-6R inhibitors (e.g., tocilizumab); type I interferon receptor antagonists (e.g., anifrolumab); BAFF inhibitors (e.g., belimumab); T-cell costimulation blockers (e.g., abatacept); and anti-integrin monoclonal antibodies (e.g., vedolizumab), as well as novel biologic constructs not yet developed at the time of filing.

To preserve structural integrity and enhance dermal or mucosal bioavailability, the biologic may be encapsulated in a stabilizing carrier system. Such carriers include solid lipid nanoparticles (SLNs), nanostructured lipid carriers (NLCs), polymeric nanoparticles (e.g., poly(lactic-co-glycolic acid) (PLGA)), depot-forming biodegradable microspheres, nanoemulsions, or liposomes. These carriers may be further stabilized using excipients such as trehalose, mannitol, polyethylene glycol (PEG), Pluronic F68, or surfactants known to enhance protein stability and solubility.

The encapsulated biologic may be embedded directly into the microneedle matrix using micromolding, centrifugal casting, or layer-by-layer fabrication, allowing for either monophasic or biphasic release profiles. The matrix material may comprise hyaluronic acid, pullulan, alginate, gelatin, chitosan, or combinations thereof. In some embodiments, the microneedles may be designed with multilayered or tip-loaded configurations to deliver the biologic with spatial or temporal control.

The microneedle system may be configured for transdermal application to areas such as the upper arm, abdomen, or thigh, or for transmucosal delivery via buccal, gingival, or sublingual sites. Delivery may be local or systemic. The system may further be designed for sustained delivery over hours, days, or weeks, depending on the degradation rate of the matrix and encapsulating carrier.

In one embodiment, the dissolvable microneedle system comprises one or more vitamins that are formulated for enhanced transdermal delivery. These vitamins may be fat-soluble or water-soluble. The DMN system enables direct delivery into the dermis, bypassing first-pass metabolism and reducing degradation, which improves stability, absorption efficiency, and therapeutic consistency. Representative examples include vitamin D3(cholecalciferol), a fat-soluble vitamin with known limitations in oral bioavailability, particularly in populations with fat malabsorption or chronic inflammatory conditions; vitamin K1 (phylloquinone) and K2 (menaquinone), which support bone health and cardiovascular function but degrade rapidly in oral or aqueous formulations; and vitamin B6 (pyridoxine), a cofactor in neurotransmitter synthesis that benefits from controlled dermal release to minimize peak-trough fluctuations. Additional examples include vitamin B1 (thiamine), which is unstable in low pH and has limited skin delivery research to date; vitamin B7 (biotin), often supplemented for hair and nail health but rarely explored in DMNs; and vitamin B5 (pantothenic acid), which plays a role in coenzyme A production and could benefit from sustained dermal delivery. Vitamin H, sometimes used interchangeably with biotin but distinguished in certain regions, and derivatives of folate (e.g., 5-methyltetrahydrofolate) are also contemplated for transdermal use in populations with MTHFR mutations. These examples are illustrative only. The present invention encompasses all known and unknown forms of vitamins, their precursors, active metabolites, derivatives, salts, or complexes, including those not yet studied, disclosed, or discovered at the time of filing.

In another embodiment, the dissolvable microneedle system comprises one or more minerals or trace minerals formulated for systemic or localized transdermal absorption. These minerals perform essential biological functions, including enzymatic catalysis, mitochondrial energy production, immune modulation, redox homeostasis, endocrine regulation, and skeletal maintenance. Incorporating minerals into a dissolvable microneedle (DMN) system addresses common limitations associated with oral mineral supplementation, such as gastrointestinal irritation, poor solubility, pH instability, and absorption inhibition by dietary compounds such as phytates, oxalates, and fiber.

Representative examples include magnesium (Mg²⁺) , a critical cofactor for over 300 enzymatic reactions, which is often poorly absorbed in its oral salt forms and may cause gastrointestinal discomfort; selenium (Se), a trace element essential for thyroid function and antioxidant defense, which is chemically sensitive in aqueous or oxidizing environments and thus benefits from stabilized dermal delivery; and chromium (Cr³⁺) , a trace mineral that supports glucose metabolism but exhibits poor bioavailability in most commercially available forms. Manganese (Mn²⁺) , involved in connective tissue formation and antioxidant defense, and boron (B), a trace mineral with emerging roles in bone metabolism, cognitive function, and hormone regulation, are also considered well-suited for transdermal microneedle delivery due to limited prior art and underexplored potential. Vanadium, which has been investigated for insulin-mimetic properties in preclinical models, has not yet been studied in transdermal systems and may represent a novel delivery application. Molybdenum, a cofactor for enzymes involved in sulfur metabolism and detoxification, and silica (Si), which contributes to connective tissue integrity and collagen synthesis.

The dissolvable microneedle system may also include calcium (Ca²⁺) , a macromineral essential for skeletal structure and neuromuscular signaling, which may exhibit impaired absorption in individuals with low gastric acid or gastrointestinal disorders. When delivered via microneedles, calcium may be combined with vitamin D or magnesium to support co-absorption and sustained bioavailability.

This is a representative list only. The invention encompasses all known and yet-to-be-discovered macro-and micro-minerals, mineral salts, oxides, chelates, and biologically active mineral complexes. It further includes combinations of these minerals with vitamins or other bioactive compounds, whether currently listed, unavailable, or not yet discovered, provided they are suitable for incorporation into a dissolvable microneedle-based delivery system.

In certain embodiments, the bioactive compound embedded in the dissolvable microneedle system may comprise one or more plant-derived actives. These include botanical extracts, essential oils, and plant-derived volatile compounds. Botanical extracts may be obtained from leaves, roots, stems, flowers, or fruits of medicinal or aromatic plants, and may include water-soluble or alcohol-soluble phytochemicals such as flavonoids, alkaloids, tannins, glycosides, saponins, and polyphenols. Essential oils and related volatile compounds may include whole oils or individual constituents such as terpenes (e.g., linalool, limonene), aldehydes (e.g., cinnamaldehyde), esters (e.g., linalyl acetate), ketones (e.g., carvone), and oxides (e.g., 1,8-cineole). These compounds may be included for their therapeutic, cosmetic, or aromatic properties, including anti-inflammatory, antimicrobial, analgesic, anxiolytic, antioxidant, or fragrance-enhancing effects. Essential oils or their constituents may optionally be encapsulated in delivery vehicles such as solid lipid nanoparticles (SLNs), nanostructured lipid carriers (NLCs), polymeric nanoparticles or any other nanocarrier prior to incorporation into the microneedle matrix to enhance stability, solubility, and controlled release. The invention contemplates the use of such plant-derived volatile compounds in transdermal, dermal, mucosal, or scalp applications, either alone or in combination with other bioactive ingredients.

In another embodiment, the dissolvable microneedle (DMN) system comprises one or more bioactive compounds derived from macroalgae and microalgae species, formulated for enhanced transdermal absorption, bioavailability, and sustained release. Algae are an abundant source of structurally diverse and biologically active compounds, many of which are sensitive to enzymatic degradation or gastrointestinal instability, making them ideal candidates for DMN-based delivery. Representative species include Spirulina (Arthrospira platensis), which is rich in essential amino acids, vitamin B12, gamma-linolenic acid (GLA), and phycocyanin, beta carotene, zeaxanthin, iron; Chlorella (Chlorella vulgaris), known for its detoxifying properties, ACE-1 inhibition and anti-inflammatory activities and high chlorophyll content; and Nannochloropsis, a microalga abundant in EPA and antioxidants. Other established algae-derived bioactives include astaxanthin from Haematococcus pluvialis, fucoidan and fucoxanthin from brown seaweeds such as Ecklonia cava and Laminaria japonica, and sulfated polysaccharides from Porphyridium cruentum, all of which are compatible with DMN formulations for metabolic, dermatologic, ophthalmic, or neuroprotective applications.

In addition to these algae species, the DMN system may incorporate bioactives from lesser-known or underutilized algae strains that have demonstrated promising pharmacological activities but have not yet been widely adopted in transdermal technologies. These include Dunaliella salina, a halotolerant microalga with one of the highest known concentrations of natural beta-carotene and zeaxanthin; Botryococcus braunii, which produces long-chain hydrocarbons, sterols, and polyunsaturated triterpenes with anti-inflammatory and antioxidant effects; and Galdieria sulphuraria, a thermoacidophilic red alga rich in glutathione and protective thiol compounds. Additional strains of interest include Skeletonema costatum, a diatom that synthesizes neuroprotective oxylipins and eicosapentaenoic acid (EPA), a key omega-3 fatty acid with anti-inflammatory and cardiovascular benefits; Scenedesmus obliquus, which contains antioxidant peptides and polyphenols; and Euglena gracilis, a flagellated microalga known for paramylon, a β-1,3-glucan with immunostimulatory activity.

The invention may also include bioactive compounds from macroalgae such as Ulva lactuca (green algae), which contains ulvans with skin-repairing properties; Codium fragile, which produces sulfated galactans with anti-allergic activity; and Sargassum fusiforme and Undaria pinnatifida, which are rich in fucoidan and phlorotannins with documented anticancer, antiviral, and anticoagulant effects. Red algae such as Gracilaria edulis and Chondrus crispus provide carrageenans with prebiotic and wound-healing potential. These species, among others, yield an array of vitamins, minerals, peptides, polysaccharides, carotenoids, lectins, polyphenols, sterols, and omega-3 fatty acids that can be encapsulated within the DMN matrix or co-formulated with lipid nanoparticles to enhance dermal penetration and systemic bioactivity.

In addition, the present invention further incorporates a broad spectrum of novel algae-derived compounds with significant potential for enhancing human health. These compounds, sourced from diverse microalgae and cyanobacteria strains, exhibit therapeutic, nutritional, and cosmetic properties and are suitable for incorporation into a DMN system for localized or systemic delivery through various delivery methods, such as oral, topical, transdermal, I..V, or any other route of administration.

Cyanobacteria, or blue-green algae, are particularly prolific producers of structurally diverse secondary metabolites. Among the notable compounds are cryptophycin 1, derived from Nostoc sp. GSV 224, a microtubule inhibitor with potent anticancer properties; and the tjipanazoles, indolocarbazole alkaloids from Tolypothrix tjipanasensis and Fischerella ambigua, which demonstrate protein kinase inhibition relevant to cancer and autoimmune therapies. Other promising bioactives include welwitindolinone A isonitrile from Hapalosiphon welwitschii, known for its neuroactivity and P-glycoprotein inhibition, and majusculamide C from Lyngbya majuscula, a cytotoxic compound with antifungal and anticancer potential. Additional compounds such as laxaphycin A and B from Anabaena, fischerellin A from Fischerella muscicola, calophycin from Calothrix fusca, and scytophycins from Scytonema pseudohofmanni display cytotoxic, antimicrobial, and anti-inflammatory activity, further supporting their utility in therapeutic and dermal applications. Bioassay-directed fractionation of the extract of the cyanobacterium P. tenue led to the isolation of the three classes of glycolipids, viz., monogalactosyl diacylglycerol (MGDG), digalactosyl diacylglycerol (DGDG), and sulfoquinovosyl diacylglycerol (SQDG) as anti-tumor-promoters.

From dinoflagellates, compounds such as amphidinol 2 from Amphidinium klebsi have shown strong antifungal properties, while yessotoxin (Dinophysis fortii) and goniodomin A (Alexandrium hiranoi) offer controlled therapeutic potential in oncology and immunology, when administered in precise doses. Okadaic acid and dinophysistoxin-1, though known as marine toxins, are powerful phosphatase inhibitors that may be repurposed under controlled delivery conditions to study or treat hyperproliferative diseases.

Bacillariophyta (diatoms) and Chrysophyta (golden algae) strains contribute polysaccharides and bioactives such as asterionellins from Asterionella sp., which may be useful in anti-aging, UV-protective, and antioxidant applications. Diatom-derived polysaccharides from Chaetoceros lauderi and Navicula delognei have also shown promise in immunomodulatory and wound-healing formulations.

Additionally, fatty acids such as r-linolenic acid from Dunaliella primolecta, Chloreococcum sp., and Stichococcus bacillaris are omega-3 compounds with known benefits in reducing inflammation, supporting cardiovascular health, and promoting skin barrier function. Compounds such as halogenated aromatics from Calothrix brevisima and cyanobacterin from Scytonema hofmanni further expand the range of bioactivities, including antibacterial and herbicidal effects, that may be incorporated into microneedle-based delivery systems for safe and effective transdermal administration.

In certain embodiments, these algae-derived bioactive compounds may be further incorporated into lipid-based or polymeric carriers such as solid lipid nanoparticles (SLNs), nanostructured lipid carriers (NLCs), or similar delivery vehicles prior to loading into the dissolvable microneedle matrix. This combination may enhance compound stability, protect sensitive bioactives from oxidation or degradation, and enable controlled or sustained release upon application to the skin. These integrated systems further support the invention's mission of delivering potent, natural, and sustainable therapeutics through minimally invasive, user-friendly formats.

This is a representative list only. The invention encompasses all known and yet-to-be-discovered algae strains and their bioactive compounds, whether microalgae, macroalgae, cyanobacteria, or extremophilic algae, and includes any bioactive derived from algae that is suitable for incorporation into a dissolvable microneedle delivery system for human or animal applications, food and beverage, cosmetic, nutraceutical, and pharmaceutical applications.

In one embodiment, the dissolvable microneedle (DMN) system comprises one or more proteins or peptides selected for their therapeutic, regenerative, cosmetic, hematologic, or immunomodulatory benefits. These macromolecules are often subject to poor oral bioavailability, enzymatic degradation in the gastrointestinal tract, and limited penetration through the skin when applied topically. The DMN platform enables localized or systemic transdermal delivery by bypassing first-pass metabolism and facilitating direct release into the dermis or interstitial space, enhancing therapeutic efficacy while minimizing degradation.

Representative examples include collagen peptides, which promote skin regeneration, wound healing, and anti-aging effects; elastin, which supports skin elasticity and structural integrity; and keratin, which strengthens the skin barrier and supports hair and nail health, particularly in scalp or dermatologic applications. Fibrin-mimetic peptides or fibrinogen, which participate in hemostasis and tissue repair, may be embedded in DMNs for wound healing or post-surgical care. In another embodiment, clotting factors relevant to hemophilia A and B, such as Factor VIII and Factor IX, may be stabilized through nanoencapsulation, polymer conjugation, cryoprotection, or equivalent method and embedded into the DMN matrix for non-invasive, sustained release. Additional examples include Spirulina-derived peptides, which possess antioxidant, anti-inflammatory, and immune-supportive properties; fibronectin and laminin, extracellular matrix proteins involved in tissue remodeling and neural repair; and phycocyanin, a pigment-protein complex from Spirulina platensis with antioxidant and anti-inflammatory effects. Due to its pH, heat, and oxidative sensitivity, phycocyanin may require nanoencapsulation or other stabilizing modifications prior to DMN integration.

In certain embodiments, proteins or peptides may require additional formulation modifications prior to microneedle embedding. These modifications may include nanoencapsulation, liposomal entrapment, PEGylation, cryoprotection, polymer conjugation, surfactant stabilization, pH buffering, or equivalent modifications to preserve structural integrity, enhance dermal penetration, improve shelf stability, and sustain post-administration bioactivity.

In another embodiment, the dissolvable microneedle (DMN) system comprises one or more lipid-based bioactive compounds, including polyunsaturated fatty acids, monounsaturated fatty acids, and carotenoids, selected for their antioxidant, anti-inflammatory, dermatologic, cardiometabolic, neuroprotective, or skin barrier-enhancing properties. These compounds are typically lipophilic and prone to oxidative degradation, limiting their bioavailability and stability in conventional oral or topical forms. The DMN platform, when combined with appropriate stabilization techniques, enables controlled transdermal delivery of these actives for both systemic and localized therapeutic effects.

Representative examples include omega-3 fatty acids such as eicosapentaenoic acid (EPA) and docosahexaenoic acid (DHA), which are known to support cardiovascular function, cognitive health, and immune modulation. Other examples include omega-6 fatty acids such as gamma-linolenic acid (GLA), which has applications in inflammatory skin conditions and hormonal regulation, and omega-9 fatty acids such as oleic acid, which supports skin hydration and barrier function. The system may also include astaxanthin, a lipid-soluble marine carotenoid with potent antioxidant activity, which is beneficial in protecting against oxidative stress in skin, neural, and ocular tissues. Due to their lipophilicity and chemical instability, these compounds may be encapsulated in solid lipid nanoparticles (SLNs), liposomes, nanoemulsions, or polymer-lipid hybrid systems prior to microneedle fabrication to ensure oxidative protection, improve dispersion, and enable sustained dermal or systemic release.

In certain embodiments, lipid-based compounds may also be co-formulated with antioxidants (e.g., tocopherols), surfactants, cryoprotectants, or pH-adjusting agents to further enhance their chemical stability and bioactivity. These stabilizing techniques may be applied before, during, or after the microneedle fabrication process to maintain compound integrity throughout processing, storage, and use.

This is a representative list. The invention encompasses all known and yet-to-be-discovered proteins, peptides, carotenoids, omega fatty acids, and other lipid-based bioactives suitable for incorporation into dissolvable microneedle systems for therapeutic, cosmetic, or nutritional applications.

In certain embodiments, the invention includes compositions comprising one or more amino acids and/or peptides stabilized and embedded within a dissolvable microneedle array for transdermal or intradermal delivery. Amino acids and peptides may be derived from natural, synthetic, or semi-synthetic sources, including algae, marine organisms, plants, fungi, microorganisms, and animal proteins. The peptides may include bioactive dipeptides, tripeptides, oligopeptides, protein hydrolysates, or modified derivatives such as esterified, acetylated, PEGylated, or ion-paired forms.

Due to the inherent susceptibility of amino acids and peptides to oxidative degradation, hydrolysis, enzymatic cleavage, and instability under certain processing conditions, the present invention employs various stabilization and encapsulation strategies prior to or during microneedle fabrication. In some embodiments, amino acids and peptides may be encapsulated within nanocarriers such as solid lipid nanoparticles (SLNs), nanostructured lipid carriers (NLCs), liposomes, niosomes, polymeric nanoparticles (e.g., chitosan, PLGA, alginate), nanoemulsions, or microemulsions. These nanocarriers may serve to protect the bioactive compounds from environmental degradation, prolong shelf-life, and facilitate controlled or sustained release following administration.

Additionally, amino acids or peptides may be lyophilized into a stable powder form prior to microneedle incorporation. Lyophilization may be carried out in the presence of one or more cryoprotectants or stabilizing agents, such as trehalose, mannitol, sucrose, sorbitol, or other polyols. The use of excipients such as antioxidants (e.g., ascorbic acid, tocopherols), metal chelators (e.g., EDTA, citric acid), enzyme inhibitors (e.g., aprotinin, leupeptin), or pH buffers may also be employed to maintain structural and functional integrity of amino acids and peptides within the matrix.

In some embodiments, the amino acid or peptide may be chemically modified into a prodrug form or conjugated to a carrier molecule via cleavable or non-cleavable linkers to enhance stability, increase lipophilicity, or improve penetration and release profiles following administration. Other techniques known in the art, including reversible masking of functional groups, site-specific conjugation, or formation of self-assembling peptide-lipid complexes, may also be employed to improve solubility, compatibility with the microneedle matrix, and bioavailability upon delivery.

The stabilized amino acids and/or peptides may be embedded within a microneedle matrix composed of biocompatible and biodegradable polymers such as hyaluronic acid, chitosan, polyvinyl alcohol (PVA), polyvinylpyrrolidone (PVP), carboxymethylcellulose (CMC), alginate, gelatin, starches, gums, or synthetic copolymers thereof. These polymers may be selected or combined based on their ability to provide mechanical strength, moisture stability, rapid or sustained dissolution profiles, and compatibility with the active ingredients and excipients. The microneedles may be designed as monolithic or multilayered structures, optionally incorporating nanocarriers into the tip or base for targeted, timed, or sequential release.

To further maintain stability during processing and storage, the microneedles may be fabricated under mild temperature and humidity conditions, and packaged in moisture-resistant, oxygen-impermeable materials, optionally including desiccants or inert gas environments. The combination of physical encapsulation, chemical stabilization, molecular modification, and optimized fabrication techniques ensures that the amino acids and peptides retain their bioactivity, structural integrity, and delivery efficacy when administered via dissolvable microneedles.

In another embodiment, the dissolvable microneedle (DMN) system comprises one or more antioxidant or anti-inflammatory compounds designed for localized or systemic transdermal delivery to address oxidative stress, inflammation, immune dysfunction, skin aging, or neurodegeneration. Many of these compounds are known to have poor oral bioavailability, rapid first-pass metabolism, or instability in gastrointestinal or topical formulations. The DMN platform enables controlled dermal penetration, bypasses digestive degradation, and supports enhanced therapeutic efficacy with improved pharmacokinetics.

Astaxanthin is a lipid-soluble marine carotenoid with potent antioxidant effects known to protect skin and ocular tissues from UV-induced oxidative stress; and epigallocatechin gallate (EGCG), a catechin from green tea with antioxidant and neuroprotective effects. Additional examples include lutein and zeaxanthin, both xanthophyll carotenoids that protect against retinal oxidative damage and are well-suited for transdermal delivery in ophthalmic and systemic applications.

The DMN system may also include phycocyanin, a pigment-protein complex extracted from Spirulina platensis, which exhibits both antioxidant and anti-inflammatory properties. Phycocyanin has been shown to inhibit COX-2, downregulate NF-κB, and activate the Nrf2 pathway, thereby reducing inflammatory cytokine expression and oxidative stress. Due to its sensitivity to light, pH, and heat, phycocyanin may be encapsulated into solid lipid nanoparticles (SLNs), liposomes, or other stabilizing carriers before being embedded into DMNs to maintain structural and functional integrity.

In certain embodiments, the DMN system may further incorporate emerging antioxidant and anti-inflammatory compounds with strong scientific support and no known prior art for microneedle-based delivery. These include apigenin, a flavonoid from chamomile that modulates GABAergic signaling and reduces pro-inflammatory cytokines; rosmarinic acid, a caffeic acid ester found in rosemary and lemon balm that protects against oxidative and inflammatory skin damage; baicalin, a flavone glycoside from Scutellaria baicalensis with neuroprotective and anti-inflammatory effects via inhibition of microglial activation and NF-κB signaling; and pterostilbene, a methylated derivative of resveratrol with enhanced bioavailability, shown to reduce inflammation and oxidative damage in vascular and brain tissue.

Other novel compounds suitable for inclusion are caffeic acid phenethyl ester (CAPE), derived from propolis and known to modulate oxidative stress in neuroinflammation; sulforaphane, a sulfur-containing isothiocyanate from broccoli sprouts that activates the Nrf2 antioxidant response pathway; boswellic acids, pentacyclic triterpenes from Boswellia serrata with clinically studied anti-arthritic and anti-inflammatory effects; andrographolide, a diterpene lactone from Andrographis paniculata with immunomodulatory and antiviral properties; and ferulic acid, a phenolic antioxidant that stabilizes other bioactives in dermatologic and anti-aging applications. All of these compounds have shown therapeutic potential across dermatologic, neurologic, metabolic, or immunologic domains, and benefit from stabilization via nanoencapsulation, emulsification, or polymer conjugation prior to DMN fabrication.

These bioactive compounds may be delivered individually or in synergistic combinations. Formulation modifications such as encapsulation in SLNs, polymeric nanoparticles, nanoemulsions, or liposomes, and co-formulation with antioxidants (e.g., tocopherols), surfactants, cryoprotectants, pH-modifying agents, or equivalent methods may be used to preserve chemical stability, enhance transdermal penetration, and control release profiles.

This is a representative list. The invention encompasses all known and yet-to-be-discovered antioxidants and anti-inflammatory compounds, including polyphenols, flavonoids, terpenoids, pigment-protein complexes, plant-derived phytochemicals, and marine-derived compounds, that are suitable for stabilization and delivery through dissolvable microneedle systems for any therapeutic, cosmetic, dermatologic, neurologic, or metabolic applications.

In one embodiment, the dissolvable microneedle (DMN) system comprises one or more neurological or psychoactive bioactive compounds formulated for controlled transdermal delivery to address mental health disorders, neurodegenerative conditions, cognitive impairment, or chronic pain. Many neurologically active compounds are limited by poor oral bioavailability, extensive first-pass hepatic metabolism, enzymatic degradation, or rapid systemic clearance. The DMN platform enables non-invasive, site-specific, and sustained delivery, improving pharmacokinetics, reducing systemic variability, and enhancing patient compliance.

In certain embodiments, bioactive compounds derived from mushrooms, such as hericenones and erinacines from Hericium erinaceus (Lion's Mane), ergothioneine from Pleurotus ostreatus (oyster mushroom), and cordycepin from Cordyceps militaris. These bioactive compounds have been associated with neuroprotective, antioxidant, and metabolic regulatory activities and may be incorporated into DMN systems.

Representative compounds include psychedelic tryptamines such as psilocybin, N,N-dimethyltryptamine (DMT), and 5-methoxy-DMT, which are currently under clinical investigation for treatment-resistant depression, post-traumatic stress disorder (PTSD), substance use disorders, and end-of-life anxiety. These compounds are rapidly metabolized by monoamine oxidase (MAO) and exhibit ultra-short half-lives when administered orally, nasally, or intravenously. Incorporation into dissolvable microneedles facilitates controlled-release delivery, bypasses enzymatic degradation, and reduces peak-trough plasma fluctuations, potentially improving therapeutic duration and tolerability. In certain embodiments, these compounds may be co-encapsulated with MAO inhibitors or stabilized using cryoprotectants, pH-modulating agents, or lipid-based nanocarriers to preserve pharmacologic activity and enhance dermal absorption.

In another embodiment, ketamine and lidocaine may be co-formulated within the DMN system to achieve dual-phase analgesia. Ketamine, an NMDA receptor antagonist, exhibits dissociative anesthesia and rapid antidepressant effects, while lidocaine provides immediate local anesthesia via sodium channel blockade. Delivery via DMNs may reduce systemic toxicity, improve tolerability, and provide both localized and systemic effects in neuropathic pain, post-surgical recovery, and treatment-resistant depression.

Cannabinoids such as cannabidiol (CBD) and tetrahydrocannabinol (THC) may also be incorporated into the DMN system for transdermal delivery in conditions involving pain, anxiety, neuroinflammation, or neurodegeneration. Due to their high lipophilicity and oxidative sensitivity, cannabinoids may be nanoencapsulated using solid lipid nanoparticles (SLNs), liposomes, or nanoemulsions prior to integration into the microneedle matrix. These strategies improve chemical stability, facilitate controlled release, and enhance skin penetration, enabling more consistent and bioavailable transdermal cannabinoid delivery.

In another embodiment, the dissolvable microneedle (DMN) system comprises one or more cognitive-enhancing, neuroprotective, or adaptogenic bioactive compounds designed for transdermal delivery to support brain function, stress resilience, memory, and mental clarity. These compounds are often limited by poor gastrointestinal absorption, extensive first-pass hepatic metabolism, or inadequate blood-brain barrier penetration, challenges that are mitigated through nanoencapsulation and microneedle-based administration.

Representative examples include citicoline (CDP-choline) and huperzine A, which enhance acetylcholine availability and synaptic plasticity and are under investigation for use in age-related cognitive decline and memory enhancement. These compounds benefit from sustained-release transdermal delivery, which avoids gastrointestinal degradation and enables more consistent plasma levels. L-theanine, a naturally occurring amino acid found in green tea, promotes relaxation by increasing alpha brain wave activity and modulating GABA and glutamate signaling. It is a promising candidate for dissolvable microneedle delivery in applications involving anxiety, cognitive stress, and sleep disturbances.

In certain embodiments, the DMN system may also incorporate adaptogenic plant extracts such as withanolides from Withania somnifera (ashwagandha), salidroside and rosavin from Rhodiola rosea, and andrographolide from Andrographis paniculata. These compounds have demonstrated the ability to reduce cortisol levels, enhance neuroendocrine balance, and improve resilience to mental and physical stress. To date, no published studies or patents have formulated these adaptogens into dissolvable microneedles, representing a novel and innovative use of DMNs for hormone-modulating, anti-fatigue, and neuroprotective applications.

In another embodiment, gamma-aminobutyric acid (GABA) and melatonin may be co-formulated into the DMN system to address circadian rhythm disruption, stress-related insomnia, and autonomic nervous system dysregulation. GABA's poor oral absorption and limited central nervous system penetration make it particularly suitable for transdermal delivery, while melatonin's short half-life is well-managed through controlled-release microneedle formats. Additionally, synergistic bioactive combinations such as melatonin+apigenin, citicoline+magnesium L-threonate, and psilocybin+sulforaphane may be embedded into DMNs to enhance therapeutic effects through multi-pathway neuroregulation, combining antioxidant, neurochemical, and neuroplasticity-supporting mechanisms.

This is a representative list. The invention encompasses all known and yet-to-be-discovered nootropics, neuropeptides, adaptogens, psychoactive compounds, phenethylamines, tryptamines, cognitive enhancers, and herbal neuroactives that are suitable for nanoencapsulation and delivery via dissolvable microneedles for therapeutic, cosmetic, or preventive applications in neurology, psychiatry, mental health, and cognitive wellness.

In another embodiment, the dissolvable microneedle (DMN) system comprises one or more bioactive compounds that regulate metabolic processes, support endocrine balance, or promote cellular energy production. These compounds may be used individually or in combination to address conditions such as insulin resistance, metabolic syndrome, hormonal dysregulation, obesity, hypothalamic-pituitary-adrenal (HPA) axis imbalance, and oxidative stress-related metabolic dysfunction. Many metabolic and hormonal bioactives exhibit poor oral bioavailability due to degradation in the gastrointestinal tract, extensive first-pass hepatic metabolism, or chemical instability. The DMN platform enables transdermal delivery of these actives in a controlled, sustained-release format, enhancing systemic absorption, reducing dosing frequency, and improving therapeutic consistency.

Representative compounds include berberine, which supports glucose metabolism and insulin sensitivity; capsaicin, a thermogenic agent that stimulates fat oxidation; and coenzyme Q10 (ubiquinone), a mitochondrial cofactor known to improve cardiovascular and cellular energy function. DHEA and phosphatidylserine may be incorporated into DMNs to support adrenal function, hormonal balance, and stress reduction, while melatonin and apigenin offer synergistic effects for circadian regulation and cortisol modulation.

In certain embodiments, the DMN system may further comprise bioactive compounds for which no prior art exists in microneedle-based delivery. These include myo-inositol, used in polycystic ovary syndrome (PCOS) and metabolic syndrome to improve insulin sensitivity and ovarian function; alpha-lipoic acid (ALA), a mitochondrial antioxidant that improves glucose utilization and nerve health; and resveratrol, a polyphenol that activates sirtuin pathways and supports anti-aging and metabolic regulation. L-carnitine, a carrier molecule for mitochondrial beta-oxidation of fatty acids, may also be delivered transdermally to enhance energy metabolism and weight management.

Additional novel compounds include 7-keto DHEA, a non-androgenic metabolite of DHEA with thermogenic and thyroid-supportive effects; pregnenolone, a precursor to multiple steroid hormones that supports mood and neuroendocrine function; and nicotinamide riboside (NR) and nicotinamide mononucleotide (NMN), both of which are NAD+precursors shown to enhance mitochondrial bioenergetics and cellular longevity. These compounds are often unstable in aqueous formulations or poorly absorbed via oral administration, making them ideal candidates for encapsulation using solid lipid nanoparticles (SLNs), liposomes, nanoemulsions, or polymer-lipid hybrid carriers prior to microneedle integration.

This is a representative list. The invention encompasses all known and yet-to-be-discovered metabolic and hormonal regulators, including vitamins, hormone precursors, plant-derived adaptogens, polyphenols, amino acid derivatives, and cofactors that can be formulated and stabilized for transdermal administration via dissolvable microneedle systems for therapeutic, wellness, or preventive health applications.

In another embodiment, the dissolvable microneedle (DMN) system comprises one or more bioactive compounds formulated for transdermal delivery to promote sleep, support nervous system relaxation, and reduce physiological or psychological stress. These compounds act through diverse mechanisms, including modulation of GABAergic and serotonergic signaling, cannabinoid receptor activation, thermoregulation, and hypothalamic-pituitary-adrenal (HPA) axis regulation. Many of these compounds suffer from poor oral bioavailability, instability in gastrointestinal environments, or undesirable systemic side effects when delivered via conventional routes. Incorporation into a DMN platform enables precise, sustained, and localized or systemic delivery while bypassing first-pass metabolism and enzymatic degradation.

Representative compounds include honokiol and magnolol from magnolia bark, which modulate GABA-A receptors and reduce cortisol; cannabinol (CBN), a mildly psychoactive cannabinoid known to promote sleep through CB1 receptor activation; and beta-caryophyllene, a non-psychoactive terpene that acts as a CB2 receptor agonist with anxiolytic and anti-inflammatory properties. Additional examples include 5-hydroxytryptophan (5-HTP), a direct serotonin precursor that improves sleep latency and melatonin synthesis; L-theanine, an amino acid that enhances alpha brain wave activity and modulates glutamate and GABA signaling; and taurine, which supports GABAergic tone and reduces sympathetic nervous system activity.

Further examples include glycine, which improves sleep quality by lowering core body temperature and modulating central nervous system activity; L-ornithine, which reduces mental fatigue and supports restorative sleep; and theobromine, a cocoa-derived compound that acts as a mild adenosine modulator with calming effects. Botanically derived bioactives such as linalool from lavender oil, valerenic acid from valerian root, matricin from chamomile, and extracts of lemon balm (Melissa officinalis) may also be incorporated for their sedative, anxiolytic, and sleep-enhancing properties. In some embodiments, triethylene glycosides and other withanolide precursors from ashwagandha (Withania somnifera) may be used to support HPA axis balance and reduce nighttime cortisol levels.

These compounds may be formulated individually or in synergistic combinations. In certain embodiments, they may be stabilized through nanoencapsulation using solid lipid nanoparticles (SLNs), liposomes, nanoemulsions, or polymer-based matrices prior to microneedle fabrication, to enhance dermal penetration, reduce oxidative degradation, and ensure sustained bioactivity. This is a representative list. The invention encompasses all known and yet-to-be-discovered natural and synthetic bioactives that promote sleep or relaxation through neurological, hormonal, or thermoregulatory mechanisms, and that are suitable for stabilization and delivery via dissolvable microneedle systems.

In certain embodiments, nanoparticles may be incorporated into the dissolvable microneedle formulation to enhance the delivery, stability, solubility, or controlled release of the active ingredient. The term “nanoparticle” as used herein refers to any carrier or encapsulation structure with a diameter generally ranging from 1 nanometer to 1,000 nanometers. Suitable nanoparticles include polymeric nanoparticles composed of biocompatible and biodegradable polymers such as poly(lactic-co-glycolic acid) (PLGA), polycaprolactone (PCL), chitosan, alginate, and polyethylene glycol (PEG); solid lipid nanoparticles (SLNs) and nanostructured lipid carriers (NLCs) composed of glyceryl monostearate, stearic acid, or mixtures of solid and liquid lipids; and vesicular carriers such as liposomes, transfersomes, niosomes, cubosomes, and ethosomes, which are composed of phospholipids and/or surfactants and may encapsulate both hydrophilic and lipophilic compounds. Additionally, micelles formed from amphiphilic block copolymers, such as PEG-PLA or PEG-PCL, may be employed to solubilize and deliver hydrophobic drugs. Cyclodextrin-based inclusion complexes may also be used to improve the solubility and stability of small molecule actives.

Further embodiments may utilize dendrimers, which are highly branched, monodisperse macromolecules capable of carrying multiple active or targeting moieties, or mesoporous silica nanoparticles (MSNs), which offer high surface area and tunable pore sizes for controlled release applications. Nanocrystals consisting of pure bioactive particles in nanoscale dimensions may be incorporated to increase dissolution rates of poorly water-soluble compounds. Protein-based nanoparticles, such as those composed of albumin, gelatin, or casein, may be used in pharmaceutical, nutraceutical, or food-grade microneedle formulations due to their biodegradability, safety profile, and compatibility with biologically active agents. Hybrid nanoparticles, including lipid-polymer hybrids and self-assembling peptide-based systems, may also be utilized to combine the structural and functional benefits of multiple delivery platforms. In some embodiments, combinations of the aforementioned nanoparticle systems may be integrated into a single microneedle formulation to achieve synergistic therapeutic, cosmetic, or nutritional effects.

The nanoparticles may be incorporated within the microneedle tip, distributed throughout the shaft, or localized within the baseplate of the microneedle patch, depending on the desired spatial release profile. The use of nanoparticles may further be combined with other technologies including, but not limited to, chemical permeation enhancers, microencapsulation systems, thermoresponsive or pH-sensitive polymers, and prodrug strategies to further optimize transdermal absorption, stability, and bioavailability. It is understood that the materials, systems, and techniques described herein are illustrative and non-limiting. Any nanoparticle-based carrier or encapsulation method, whether currently known, modified, functionally equivalent, or subsequently developed, may be used within the scope of the invention to enable or enhance delivery through dissolvable microneedle systems across pharmaceutical, cosmetic, nutraceutical, veterinary, and agricultural applications.

In certain embodiments, microencapsulation techniques may be utilized in the fabrication of dissolvable microneedle formulations to protect the active ingredient, improve its physical and chemical stability, enhance solubility, and modulate its release profile following administration. Microencapsulation refers to the process of enclosing active compounds within microscale carriers or matrices—typically ranging from 1 to 1,000 microns in diameter—using natural, synthetic, or semi-synthetic materials. These microcapsules or microspheres may be incorporated directly into the microneedle matrix, localized in the tip, or distributed throughout the baseplate depending on the desired delivery kinetics.

Various microencapsulation methods may be employed, including but not limited to spray drying, spray chilling, coacervation (simple or complex), solvent evaporation, ionic gelation, interfacial polymerization, fluidized bed coating, freeze-drying, and electrospray encapsulation. Each technique may be selected based on the physicochemical characteristics of the active ingredient, the intended release profile, and compatibility with the microneedle-forming polymers. Suitable encapsulating materials include gelatin, alginate, chitosan, gum arabic, poly(lactic-co-glycolic acid) (PLGA), ethyl cellulose, polyvinyl alcohol (PVA), lipids, and food-grade starch derivatives, or any other natural, synthetic, or semi-synthetic encapsulating materials that are biocompatible, biodegradable, or otherwise capable of forming microcapsules or microspheres suitable for incorporation into dissolvable microneedle systems, including materials not explicitly listed herein or not yet discovered at the time of this filing.

Microencapsulation may serve to prevent degradation of moisture-sensitive or thermolabile actives during manufacturing and storage, enable sustained or controlled release after skin insertion, reduce local irritation, mask color and taste in nutraceutical or food-grade applications, or enable combination therapy through co-loading of multiple agents. These systems may be incorporated as powders, dispersions, or suspensions into the microneedle casting solution, and may be used in conjunction with other enhancement strategies, including nanoparticle systems, permeation enhancers, prodrugs, or stimuli-responsive polymers.

The microencapsulation systems and methods described herein are intended to be illustrative and not limiting. Any functionally equivalent microencapsulation technique or formulation—including those not explicitly listed, not yet developed, or subsequently discovered—may be used within the scope of the invention, provided that it supports the desired release, protection, or performance of the active ingredient within a dissolvable microneedle system. This includes but is not limited to applications in pharmaceutical, cosmetic, nutraceutical, and functional food delivery platforms.

In certain embodiments, the dissolvable microneedle formulation may optionally include preservatives or excipients to enhance formulation stability, processing characteristics, mechanical strength, or microbial safety. Preservatives such as phenoxyethanol, benzyl alcohol, sodium benzoate, or naturally derived antimicrobial agents may be incorporated when the formulation contains water or moisture during manufacture or is intended for prolonged storage under variable conditions. In other embodiments, the microneedle formulation may be free of preservatives, particularly when the product is dried thoroughly and stored in moisture-barrier packaging. Excipients such as polyvinyl alcohol, carboxymethylcellulose, glycerol, trehalose, mannitol, or antioxidants may also be included to improve moldability, dissolution rate, mechanical strength, or active compound stability. The use or omission of such additives may be determined based on the intended application, target industry (pharmaceutical, cosmetic, nutraceutical, or food), and regulatory considerations. Equivalent materials and future excipient systems performing similar functional roles are also considered within the scope of this invention.

In certain embodiments, additional strategies may be employed in conjunction with dissolvable microneedle systems to enable responsive, targeted, or enhanced delivery of active agents. Thermoresponsive or pH-sensitive polymers, such as poly(N-isopropylacrylamide) (PNIPAAm), Eudragit, and chitosan derivatives, may be integrated into the microneedle matrix to enable environment-responsive drug release triggered by changes in temperature or pH at the site of application. Enzyme-responsive materials may also be incorporated into dissolvable microneedles to enable selective degradation or activation in response to enzymes such as esterases or proteases, particularly within inflamed or tumor-associated microenvironments. Layer-by-layer (LbL) assembly techniques may be used to construct microneedles with precisely structured multilayered cores or surface coatings, allowing for sequential or controlled release of multiple agents. Ionic liquids and deep eutectic solvents may serve as biocompatible solubilizers or permeation enhancers to facilitate the incorporation of poorly soluble actives into the microneedle formulation. Advanced 3D printing technologies, including digital light processing (DLP) and two-photon polymerization, may be used to fabricate customized microneedle geometries, internal drug reservoirs, or multicomponent patch systems with high precision and reproducibility. Bioadhesive and mucoadhesive polymers, such as polyacrylic acid, Carbopol, or chitosan, may be used to enhance microneedle adherence to wet biological surfaces such as the oral mucosa or conjunctiva, expanding the use of DMNs beyond dermal applications. In other embodiments, microneedles may be co-formulated with nanobubbles or gas-generating compounds, which can enhance active dispersion, penetration, or local diffusion through the creation of mechanical forces upon dissolution. Magnetically responsive particles may also be incorporated to enable external control over microneedle localization, release behavior, or therapeutic targeting through the application of magnetic fields.

These methods are illustrative and not limiting. Any other delivery-enhancement or formulation-modification technique, whether currently known, adapted, functionally equivalent, or developed after the time of filing, may be employed in combination with dissolvable microneedle systems within the scope of this invention.

### Structure of Dmn System

It is understood that the delivery system of the present invention does not have to be a patch, but may be implemented in various other formats, including dissolvable microneedle films, strips, swabs, rollers, or wearable textile-integrated devices, provided that such configurations facilitate dermal, mucosal, or transcutaneous delivery through microneedle penetration and dissolution. FIG. 1 shows a diagram of a DMN system. As shown, each microneedle is formed as a solid conical, pyramid, or hybrid geometrical shape structure configured to dissolve upon contact with interstitial fluid in the skin. In certain embodiments, the microneedles may have a tapered or multi-segmented structure to support staged dissolution or differential payload release. The height-to-base aspect ratio may range from 1.5:1 to 5:1, preferably between 2:1 to 4:1 to optimize penetration efficacy while minimizing mechanical stress and fracture risk during insertion. The height of each microneedle is preferably 500 micrometers (μm) to allow it to penetrate into the dermis so that the bioactive compound on the microneedle or within the interior of the microneedle could be released into the skin's interstitial space. However, the microneedle height could also range from 150 micrometers to 2000 micrometers, depending on anatomical site, skin thickness, or therapeutic application. For instance, 300-500 μm microneedles may be used for shallow intradermal applications such as cosmetic or dermatologic treatments, while longer microneedles (700-1000 μm) may be used for systemic or deeper dermal delivery. In one embodiment, penetration depths ranging from 200 μm to 600 μm are considered suitable for delivery to the dermis without reaching vascular or nerve-dense regions. In another embodiment, skin penetration depths from approximately 262 μm to 469 μm are acceptable for transdermal delivery.

Dissolvable microneedles (DMNs) may be fabricated in a variety of shapes, each selected to optimize mechanical strength, skin insertion efficiency, dissolution behavior, and delivery of bioactive compounds. Commonly used geometries include conical microneedles, which are tapered to a sharp point to facilitate effective penetration of the stratum corneum with minimal discomfort. Pyramidal microneedles possess a square or triangular base that converges to a pointed apex, providing enhanced structural rigidity and uniform drug delivery. Funnel-shaped microneedles exhibit a wider upper region that tapers sharply toward the tip, potentially modifying insertion depth and dissolution kinetics. Candlelit-shaped microneedles incorporate a narrowed waist and flared base, offering distinct insertion and breakage profiles. Barbed microneedles, which feature micro-scale protrusions or backward-facing angles along the shaft, are designed to increase skin adhesion and enhance retention during drug delivery. Additional geometries include cylindrical, blade-like, chisel-tipped, and hemispherical-tipped microneedles, each offering specific advantages for penetration force, dissolution rate, or fabrication compatibility. The geometry may also be asymmetric or hybrid in nature, combining features of multiple shapes to address application-specific needs such as multi-layer drug loading, unidirectional insertion, or tissue targeting. Furthermore, the microneedles may incorporate novel designs that have yet to be developed or disclosed. The microneedle shape may be optimized based on intended therapeutic indication, target patient population, skin type, or material composition, and the shapes described herein are not limiting.

In certain embodiments, the microneedles may exhibit a barbed geometry. As used herein, “barbed” refers to microneedle structures that include one or more backward-facing projections, ridges, or hook-like features along the shaft or near the tip. These structural elements may be designed to resist withdrawal from tissue, promote retention at the site of insertion, and enhance contact duration with skin or mucosa. Barbed geometries may be especially beneficial for applications requiring extended residence time, localized delivery, or where mechanical retention is desired, such as in cosmetic facial patches, veterinary applications, or agricultural foliar delivery.

The microneedle base diameter or width may range from 100 μm to 500 μm, depending on the needle height, intended dose, and material composition. In one embodiment, the base diameter is between 150 μm and 300 μm. The cross-sectional thickness of the microneedle body is preferably between 20 μm and 80 μm. Thinner microneedles (20- 40 μm) may be desirable for rapid-dissolving, single-dose applications, while thicker microneedles (50-80 μm) may be used to support multi-layered, controlled-release systems or high molecular weight actives.

The spacing between microneedles on the patch array may range from 300 μm to 1200 μm center-to-center. Denser arrays (e.g., 300-500 μm spacing) enable higher delivery surface area and greater dose per application for small-molecule or low-dose actives, whereas wider spacing (800-1200 μm) may be used to reduce skin irritation, accommodate larger drug volumes per needle, or allow for actives that require diffusion across broader dermal fields.

The total number of microneedles per patch may vary based on therapeutic need, ranging from fewer than 10 microneedles for targeted microdosing applications to greater than 1000 microneedles in high-density patch configurations. In some embodiments, patches may be modular or segmented to allow for application to multiple body sites or delivery of distinct compounds from different regions of the same patch.

Each microneedle is composed of one or more biocompatible, biodegradable, and water-soluble polymers or polysaccharides that fully dissolve in skin interstitial fluid without leaving particulate residue.

Suitable materials for the fabrication of dissolvable microneedles include polymers from the following categories: natural polysaccharides (e.g., hyaluronic acid, sodium alginate, carboxymethyl cellulose), proteins and protein-based biopolymers (e.g., gelatin, silk fibroin, collagen), synthetic water-soluble polymers (e.g., polyvinyl alcohol, polyvinylpyrrolidone, polyethylene glycol), and semi-synthetic cellulose derivatives (e.g., methylcellulose, carboxymethylcellulose, hydroxypropyl methylcellulose, starch), as well as any equivalent or functionally similar materials that are biocompatible, biodegradable, water-soluble or water-swellable, and capable of forming mechanically robust microneedles that dissolve fully in interstitial fluid. This includes materials not yet known or developed at the time of filing, provided they meet the performance criteria required for microneedle fabrication, insertion, and dissolution for transdermal or intradermal delivery of active agents.

In certain embodiments, two or more natural biopolymers may be combined in the same formulation to fabricate microneedles within a single mold. For example, hyaluronic acid may be blended with chitosan, pullulan, or alginate to tailor the microneedle's mechanical properties, dissolution rate, or compatibility with specific bioactives. Such combinations allow for structural customization, biphasic release, or improved skin insertion performance. These natural polymer blends may also be optionally crosslinked to further control degradation rate, moisture sensitivity, and long-acting functionality. The microneedles may be fabricated using a single-cast or layer-by-layer molding technique, with uniform or stratified distribution of the polymers throughout the microneedle shaft or tip.

These materials may be used individually or in combination to achieve critical performance parameters of the microneedle matrix, including, but not limited to, mechanical strength, insertion force, structural integrity, dissolution rate, hydration responsiveness, compatibility with active compounds, and overall biocompatibility and safety for transdermal or intradermal delivery. The selection and ratio of base materials may be adjusted to accommodate a wide variety of formulation needs, including compounds that are thermolabile, hygroscopic, pH-sensitive, hydrophobic, or high-molecular-weight macromolecules.

In some embodiments, the microneedle formulation may further comprise one or more excipients selected from the group consisting of stabilizers, plasticizers, solubilizers, dispersants, disintegrants, humectants, antioxidants, or buffering agents, depending on the physicochemical properties of the embedded bioactive compound(s). Non-limiting examples of suitable excipients include trehalose, a disaccharide that functions as a cryoprotectant, stabilizing proteins and peptides during drying and improving the rehydration response upon skin contact; glycerol and polyethylene glycol (PEG), which serve as plasticizers to enhance matrix flexibility and reduce brittleness; mannitol and sorbitol, which improve flowability and structural uniformity during casting or molding processes; lecithin and phospholipids, which may function as emulsifiers or surfactants for lipophilic compounds; ascorbic acid, tocopherols, or other antioxidants, which may be added to reduce oxidative degradation of sensitive actives; and citric acid, sodium citrate, or other buffering agents, which may be included to modulate pH during microneedle formation or dissolution.

In certain embodiments, nanoparticle-stabilizing agents such as poloxamers, Tween 80, or Pluronic F68 may be used to preserve the structural integrity of nanoencapsulated bioactives embedded within the microneedle matrix. Additional formulation aids, such as surfactants, crosslinkers, or mucoadhesive polymers, may also be employed depending on the application-specific requirements, particularly in hybrid systems or dual-layer microneedles designed for multi-phase release.

These excipients may be present in concentrations ranging from 0.1% to 50% by weight of the dry microneedle formulation, depending on their function, interaction with the matrix material, and compatibility with the active compound. The inclusion of such excipients ensures uniform compound distribution, reproducible mechanical properties, and consistent dissolution profiles, thereby enabling scalable and effective microneedle-based delivery of a broad spectrum of bioactive agents.

The mechanical strength of each microneedle is preferably sufficient to pierce the stratum corneum and upper layers of the epidermis without bending, cracking, or breaking. Each microneedle has a minimum fracture force of greater than 0.058 N to ensure that they can penetrate the skin effectively. Insertion forces may range from 0.1 N to 3.0 N per microneedle, allowing for effective skin penetration through manual application without the need for external insertion devices. Each microneedle is preferably designed to dissolve within a predefined time window ranging from a few seconds to several hours, depending on the composition, bioactive compound, application site, and therapeutic objective. In certain embodiments, the microneedle may dissolve within 1 to 60 minutes, while in other embodiments, the dissolution may occur in less than 1 minute for rapid release, or extend beyond 60 minutes for sustained or layered delivery profiles.

In some embodiments, the present invention provides DMNs with an insertion force of at least 0.058 N per microneedle, which is the minimum force generally required for a single microneedle to pierce the stratum corneum of human skin or a validated skin simulant such as pig skin, silicone rubber, or Parafilm®M. The microneedles are preferably designed to resist mechanical fracture up to a force of approximately 0.38±0.10 N per microneedle, although the actual fracture threshold may vary depending on the needle geometry, polymer composition, and fabrication method. Insertion forces for the full microneedle patch, as applied by hand, are preferably within the range of 0.1 to 3.0 N per patch, enabling manual application without specialized devices. These values ensure that the microneedles can reliably penetrate the skin without structural failure, while maintaining user comfort and ease of application.

Various biocompatible and water-soluble materials may be used in the fabrication of dissolvable microneedles, selected based on their mechanical integrity, dissolution rate, process compatibility, and safety for transdermal administration. Natural polymers commonly employed include hyaluronic acid (HA), chitosan, carboxymethyl cellulose, gelatin, dextran, starch and modified starches such as amylopectin, pullulan, alginate, and silk fibroin. These materials are biodegradable and capable of forming needle structures that dissolve upon insertion into the skin. In addition to natural polymers, synthetic and semi-synthetic materials are frequently used to improve structural strength and control release profiles. These include polyvinylpyrrolidone (PVP), polyvinyl alcohol (PVA), polyethylene glycol (PEG), hydroxypropyl methylcellulose (HPMC), maltodextrin, and amine-functionalized polymethacrylates such as Eudragit E. Formulations may also incorporate functional excipients such as sodium polyacrylate to adjust swelling or dissolution characteristics. The selected materials must be capable of forming microneedle arrays with sufficient mechanical strength to penetrate the stratum corneum and subsequently dissolve in interstitial fluid to release the incorporated therapeutic agent. The above list is not intended to be limiting, and additional materials that are biocompatible, water-soluble, and capable of supporting the fabrication and functional performance of dissolvable microneedles, including materials not yet identified or commercially available at the time of this application, may also be utilized.

In certain embodiments, these polymers may be combined or modified to enhance performance characteristics such as dissolution rate, insertion strength, flexibility, and compatibility with hydrophilic, hydrophobic, or biologically sensitive active compounds. The invention further encompasses any currently unknown or yet-to-be-developed polymers, copolymers, derivatives, or polymer blends that meet the structural and functional criteria for use in dissolvable microneedles, including those derived from natural, synthetic, semi-synthetic, or bioengineered sources. These may include materials designed or optimized in the future for enhanced loading capacity, controlled release, improved dermal permeation, or environmental stability, provided they are capable of forming microneedles that are safe, effective, and fully dissolvable within the skin.

While synthetic materials may be used for DMN fabrication, a naturally occurring compound is more desirable for health reasons. The selection of materials for dissolvable microneedles is critical, as synthetic or non-biocompatible polymers can lead to adverse health outcomes. For example, DMNs fabricated using non-degradable synthetic polymers such as polyacrylamide or certain methacrylates have been associated with localized skin inflammation, delayed dissolution, and polymer residue accumulation in subcutaneous tissue. In some cases, patients experienced prolonged erythema or allergic contact dermatitis due to the presence of residual monomers or chemical crosslinkers not fully removed during fabrication. Such reactions not only compromise patient safety but also reduce therapeutic efficacy and undermine regulatory approval. To avoid these issues, dissolvable microneedles are increasingly formulated using natural, biocompatible, and FDA-recognized excipients like hyaluronic acid, carboxymethyl cellulose, and polyvinyl alcohol, which dissolve completely and safely after administration. Hyaluronic acid and pullulan are highly suitable for this application. Both are generally regarded as safe (GRAS) by the FDA and widely used in transdermal biomedical applications. Hyaluronic acid is a naturally occurring molecule in the body that plays a crucial role in maintaining hydration, elasticity, and tissue repair. It is widely utilized in medical and cosmetic products to enhance skin moisture, support joint function, and promote healing, and it has been utilized by several research laboratories for the fabrication of microneedles. Pullulan is a high molecular weight polysaccharide (200-300 kDa), that is commonly used to prepare DMN's film-forming properties. Pullulan is an ideal material for DMNs due to its excellent film-forming properties, biocompatibility, biodegradability, hydrophilicity, adhesiveness, transparency, flexibility, stability, and compatibility with a wide range of active ingredients. To prepare the DMNs, mixtures containing either hyaluronic acid or pullulan are centrifuged at 10,000 rpm for 5 minutes and subjected to vacuum (1 mbar) for 4 hours. The resulting dispersions will be poured into PDMS molds and subsequently dried.

In addition to hyaluronic acid and pullulan, a variety of other naturally derived polymers may be employed in the fabrication of dissolvable microneedles. These materials are selected for their biocompatibility, biodegradability, film-forming capacity, and mechanical strength sufficient to support skin insertion without fracture. Examples include chitosan, a cationic polysaccharide derived from chitin, which offers antimicrobial properties and mild mucoadhesiveness. Carboxymethyl cellulose (CMC), a cellulose derivative, is water-soluble and forms stable, high-viscosity solutions suitable for micromolding applications. Gelatin, a protein obtained from partial hydrolysis of collagen, is widely used for its film-forming ability and thermosensitive gelation properties. Dextran, a branched polysaccharide composed of glucose units, exhibits rapid dissolution and is well-suited for applications requiring fast drug release. Starch-based polymers, such as amylopectin or chemically modified starches, may be used to enhance mechanical rigidity and drying performance. Alginate, derived from brown algae, may be incorporated alone or blended with other materials to influence dissolution and gelation behavior. Silk fibroin, a protein extracted from silkworm cocoons, is recognized for its high mechanical strength, biocompatibility, and ability to support sustained-release applications in microneedle form. These naturally derived materials may be used independently or in combination, with or without additional excipients, to optimize the performance of dissolvable microneedle systems for specific therapeutic or cosmetic applications. Any biocompatible, biodegradable, and water-soluble polymer capable of forming mechanically stable microneedle structures that dissolve upon skin insertion may be used. This includes materials not specifically disclosed or yet to be developed.

In certain embodiments, the matrix material used to fabricate dissolvable microneedles may be derived from bioactive compounds extracted from naturally occurring and sustainably sourced algae. These compounds include fucoidan, laminarin, porphyran, xylan, mannan, lectins, and phlorotannin, which are derived from various brown, red, or green algae species. These algae-based compounds are abundant in marine environments and can be harvested or cultivated with minimal ecological impact, supporting the development of environmentally responsible biomedical technologies. In addition to their sustainable origin, these bioactive compounds exhibit characteristics that are desirable for DMN fabrication, including biocompatibility, water solubility, film-forming ability, hydrogel-forming capacity, and structural integrity upon drying. These functional attributes suggest that such materials may serve as natural and novel alternatives to synthetic or conventionally used polymers for the formation of dissolvable microneedle arrays. The invention further includes the use of additional algae-derived compounds not explicitly listed herein, including those not yet commercially available, fully characterized, or discovered at the time of filing, provided such materials exhibit functional suitability for dissolvable microneedle fabrication.

The fabrication of microneedles with insufficient mechanical strength is a common challenge. In an embodiment, composite materials can be used to increase the mechanical strength of the microneedles. Chitosan and carboxymethylcellulose may be incorporated into the formulations of the DMNs to improve mechanical strength. Chitosan, a naturally derived polymer produced from chitin (a widely distributed polysaccharide found in nature) is traditionally prepared via deacetylation of chitin, a chemical process that removes acetyl groups and substitutes them with reactive amino groups. Carboxymethylcellulose (CMC), a carboxymethylated derivative of cellulose, also improves the mechanical strength of DMNs. It is biocompatible, biodegradable and hygroscopic, non-toxic, and easily dispersible in water.

In addition, other materials such as polyvinyl alcohol (PVA), gelatin, starch derivatives, and silk fibroin may also be utilized, either independently or in combination with weaker film-forming polymers such as hyaluronic acid or pullulan. These materials can improve the mechanical performance of microneedles by increasing elasticity, stiffness, and resistance to deformation during insertion. The use of crosslinking agents or cryogenic processing techniques may further enhance microneedle strength without compromising biocompatibility or dissolution characteristics. The selection and proportion of strengthening agents may be tailored based on the physical properties of the target polymer matrix, desired insertion depth, and payload characteristics. The invention is not limited to the materials disclosed herein and may include any suitable polymeric additive or formulation approach capable of improving mechanical strength.

In certain embodiments, the dissolvable microneedles (DMNs) may be fabricated with multi-layered, tip-loaded, or compartmentalized architectures to enable spatially or temporally controlled release of one or more embedded bioactive compounds. For example, the microneedle tip may contain a rapidly dissolving layer for immediate release, while the base or shaft contains a slower-dissolving layer for sustained or delayed delivery. All layers are composed of biocompatible and biodegradable materials that fully dissolve in skin interstitial fluid without leaving residue.

In some embodiments, the microneedle array may be configured in various geometric layouts, including square, rectangular, circular, or hexagonal patterns, with patch sizes ranging from approximately 0.5 cm² to 10 cm² and microneedle counts ranging from fewer than 10 to over 1000 per patch, depending on the application, dose, and anatomical site. Microneedle spacing, height, and density may be optimized to accommodate skin properties and the physicochemical characteristics of the active compound.

Each DMN patch includes a backing layer, which may be composed of flexible, medical-grade films such as polyurethane, polyethylene, or hydrocolloid composites. In certain embodiments, the backing layer may include an adhesive border, peelable liner, or occlusive element to facilitate secure application and moisture retention during insertion and dissolution.

Dissolvable microneedles may be manufactured using techniques such as micromolding, centrifugal casting, layer-by-layer casting, or solvent-based deposition, allowing for reproducible formation of consistent needle geometries, embedded payloads, and programmable dissolution profiles. Dissolution times may range from a few seconds to over one hour, depending on the selected polymers, plasticizers, and structural configuration. In preferred embodiments, the microneedles dissolve fully within 1 to 30 minutes, though faster or slower profiles may be used based on therapeutic goals.

This invention encompasses all structural and material variations of dissolvable microneedle systems that are designed to penetrate the skin, dissolve in interstitial fluid, and release embedded active agents in a controlled and biocompatible manner. This includes presently known and yet-to-be-discovered biopolymer systems, fabrication strategies, and design variations that meet the performance criteria for use in dissolvable microneedle delivery platforms.

The dissolvable microneedle (DMN) systems described herein may be fabricated in a wide variety of shapes, sizes, geometries, and configurations to accommodate diverse anatomical locations, therapeutic indications, and user needs. The microneedle arrays may be formed as standalone patches, modular sections, flexible wraps, or wearable devices and may be adapted for use on localized or large surface areas of the human or animal body. In certain embodiments, the DMNs may be shaped for targeted delivery to specific anatomical sites, such as crescent-shaped arrays for under-eye application, circular or dot-shaped arrays for spot treatments (e.g., acne or blemishes), linear strips for application to the jawline or gumline, or contoured pads conforming to the cheek and temporal region for conditions such as Bell's palsy. Spot-treatment patches may range from approximately 2 mm to 15 mm in diameter, gumline strips may vary from 3 mm to 15 mm in width and 1 cm to 12 cm in length, and facial DMN patches may range from 1 cm to 8 cm in width and 2 cm to 15 cm in length. Larger therapeutic patches or wraps for conditions such as sciatica or full-body wellness may range from 5 cm to over 50 cm in length, and from 2 cm to 30 cm in width, including formats such as flexible lumbar strips, torso wraps, or multi-zone therapeutic sheets.

The microneedles themselves may vary in height from approximately 100 μm to 2,000 μm, with base widths ranging from 20 μm to 500 μm, depending on the application site, penetration depth desired, drug payload, and skin type. The microneedle density may range from 10 to 1,000 needles per square centimeter, and the overall patch or device thickness may vary from 100 μm to several millimeters, depending on design and therapeutic load. Ergonomic and aesthetically inspired shapes such as heart-shaped, teardrop, triangular, wave-form, grid-like, or modular interlocking forms may also be employed to improve fit, coverage, or user engagement.

The shape, size, and form factor may be selected or customized based on the intended therapeutic target, including facial nerves, spinal nerves, oral tissues, joints, muscle groups, or generalized skin areas. While specific configurations and dimensions are described herein, it is understood that these are provided for illustrative purposes only and are not intended to limit the invention. The scope of the invention includes all current and future microneedle array geometries and dimensional profiles capable of supporting localized, systemic, or wellness-based transdermal delivery using a dissolvable microneedle system.

In certain embodiments, the dissolvable microneedle (DMN) system may include one or more embedded indicators configured to provide real-time or delayed feedback regarding the extent of compound delivery, microneedle dissolution, or treatment completion. These indicators may be visual, chemical, physical, or sensory in nature, and may be designed to communicate information directly to the user, a caregiver, or a device. Examples include, without limitation, colorimetric indicators, opacity changes, fluorescence, visible residue, scent emission, tactile changes, or temperature shifts that correspond to the release profile or degradation status of the DMN formulation.

In one embodiment, the DMN may include a color-changing dye or pigment that transitions as the active agent is released, providing a visual cue of progress or completion. In another embodiment, the indicator may emit or alter a fluorescent or optically detectable signal that can be captured by a smartphone, wearable scanner, or remote sensor, with output interpreted by an application or software interface to determine drug depletion, timing of reapplication, or adherence tracking. The detection may be manual (e.g., by eye) or automated (e.g., using smartphone cameras, spectrometers, or image analysis algorithms). Machine learning models may also be employed to correlate visual or sensory changes with pharmacokinetic profiles or individualized delivery patterns.

The indicator may be integrated into the microneedle matrix, the patch backing, or a separate detection layer, and may be co-encapsulated with the drug, physically separate, or chemically responsive to local changes (e.g., hydration, pH, enzymatic activity, interstitial fluid composition, body heat, or electrical resistance). The invention is not limited to colorimetric or optical approaches and includes all current and future-discovered indicator technologies capable of signaling compound depletion, user compliance, or delivery status in a dissolvable microneedle system. This includes systems for human or veterinary use, clinical or at-home care, and standalone or device-integrated monitoring systems.

In certain embodiments, the dissolvable microneedle system may further include biosensor elements, either integrated into the microneedle matrix or co-deposited on the patch backing, configured to monitor interstitial biomarkers (e.g., glucose, cortisol, inflammation markers), pH, temperature, or enzymatic activity. These biosensors may enable real-time feedback regarding therapeutic delivery, physiological status, or user compliance. The biosensor signal may be detected visually (e.g., colorimetric change) or electronically via a mobile or wearable interface.

### Manufacturing of Dmn System

A variety of fabrication methods are specifically applicable to the production of dissolvable microneedles (DMNs), which are composed of biodegradable or water-soluble materials that fully dissolve upon insertion into the skin. These methods are selected based on the physical properties of the materials, the type of active agent being incorporated, and the intended clinical application. The most commonly employed approach is micromolding, in which a drug-polymer solution is introduced into microneedle-shaped molds and subsequently dried or cured to form solid microneedle structures. This technique may be conducted using gravity, vacuum, or centrifugal force to ensure complete cavity filling and uniform geometry. Piezoelectric dispensing is another DMN-specific method that enables precise volumetric control when depositing small quantities of polymer-drug solution into mold cavities. This is particularly advantageous when working with sensitive biological agents or when fabricating multilayer microneedles. Variations of molding, including centrifugal molding and the cascade microneedle molding technique (CMMT), offer scalable, low-cost alternatives for mass production.

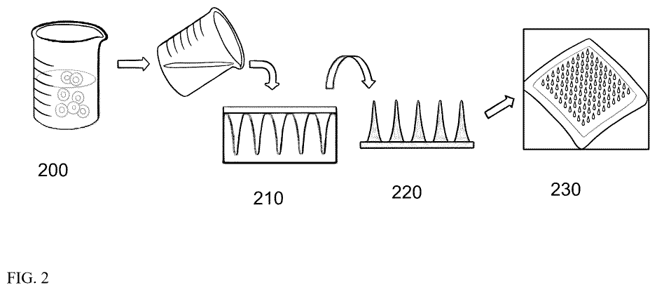

FIG. 2 shows a diagram of a micromolding process for an embodiment of the present invention. A mixture of a bioactive compound and a polymer is created 200. It is then introduced into a mold 210, dried and demolded 220, and a backing layer is added 230 to create a DMN system.

Several mold-free techniques have also been developed specifically for the fabrication of dissolvable microneedles. Centrifugal lithography, for example, uses centrifugal force to shape polymer droplets into microneedle geometries without requiring a mold. Drawing lithography forms microneedles by extending a polymer solution droplet during its viscoelastic transition phase. Both techniques enable fine control over microneedle architecture using only biodegradable materials. Droplet-born air-blowing is another mold-free technique in which an air stream elongates viscoelastic polymer droplets to form microneedle structures, offering a scalable approach without the need for molds. Additive manufacturing technologies, such as 3D printing and photopolymerization, may be employed when using photoreactive or printable dissolvable materials, allowing for dissolvable microneedle designs and geometries with high spatial resolution. Photopolymerization directly cures light-sensitive biodegradable polymers into dissolvable microneedles using patterned UV or visible light, distinguishing it from photolithography, which is typically used to create molds. In some cases, 3D-printed molding combines the speed and flexibility of additive manufacturing with the reproducibility of micromolding by using printed master molds to shape dissolvable microneedles. Equivalent or hybrid techniques that utilize variations of these core principles, such as jetting, extrusion-based printing, or light-assisted drawing, that are under development or not yet developed may offer similar benefits in terms of precision, scalability, or biocompatibility.

Atomized spray deposition has been adapted to fabricate dissolvable microneedles by spraying the polymer-drug solution into molds, improving filling consistency for high-viscosity formulations. Microfluidic drop dispensing represents a precision method for spatially controlling the distribution of polymers and active ingredients in microneedle arrays. Additionally, freeze-drying has been applied in combination with mold-based fabrication to produce porous microneedle structures, enhancing drug loading and dissolution rates. Each of these techniques is applicable to dissolvable microneedles and relies on polymers such as hyaluronic acid, polyvinylpyrrolidone (PVP), polyvinyl alcohol (PVA), carboxymethyl cellulose (CMC), or other biodegradable matrices that are compatible with transdermal delivery and complete in vivo dissolution.

The micromolding technique is a widely utilized, cost effective, and reproducible method for fabricating dissolvable microneedles intended for transdermal delivery of therapeutic agents. The process begins with the creation of a master mold, which defines the geometry of the microneedles. These master molds are fabricated using precision techniques such as micromilling, photolithography, or high-resolution three-dimensional (3D) printing. From the master mold, a negative production mold is formed, commonly using an elastomeric material such as polydimethylsiloxane (PDMS), to replicate the microstructures with high fidelity. Once the production mold is prepared, it is filled with a liquid formulation composed of one or more water-soluble or biodegradable polymers, such as hyaluronic acid, polyvinyl alcohol (PVA), or carboxymethyl cellulose (CMC), in combination with the desired active ingredient. Filling may be achieved through vacuum-assisted molding, centrifugation, or positive-pressure microperfusion to ensure the cavity has been completely occupied and to minimize air bubble entrapment. Following filling, the mold is subjected to a drying process under controlled environmental conditions to solidify the formulation. Drying methods may include air drying, spin-casting, or lyophilization, depending on the thermal sensitivity and solubility characteristics of the materials used. Once the formulation is fully solidified, the microneedles are carefully demolded to preserve structural integrity and sharpness. The flexibility and hardness of the mold material are selected to support high demolding efficiency and preservation of needle geometry. Final quality assessment includes evaluation of microneedle shape, dimensional accuracy, mechanical strength, and skin insertion capability. This micromolding approach enables the scalable production of dissolvable microneedles with consistent performance characteristics and is suitable to deliver a wide range of bioactive compounds.

Various mold materials may be employed in the fabrication of dissolvable microneedles, provided they are compatible with aqueous, biodegradable polymer formulations and capable of replicating microscale features with high fidelity. The most widely used mold material is polydimethylsiloxane (PDMS), a silicone-based elastomer known for its biocompatibility, flexibility, and ability to accurately reproduce the geometry of microneedle arrays. PDMS molds are typically produced by casting the uncured elastomer over a rigid master mold and curing it to form a reusable negative mold. Their elastomeric properties facilitate efficient demolding without damaging the delicate needle structures, making them particularly suitable for repeated use in both laboratory and pilot-scale manufacturing. In addition to PDMS, other elastomeric or thermoplastic materials, such as polyurethane (PU) and thermoplastic elastomers (TPE), may be utilized when greater durability or dimensional stability is required. These materials can withstand mechanical stress during filling and demolding while maintaining mold fidelity. In limited applications, particularly during prototyping, rigid thermoplastics such as polystyrene (PS) and polycarbonate (PC) may be used to fabricate single-use molds. While these offer dimensional precision, their rigidity can present challenges during demolding, potentially increasing the risk of microneedle damage. More recently, high-resolution three-dimensional (3D) printed molds fabricated from biocompatible photopolymer resins have been employed to enable custom microneedle designs, particularly in early-stage research and development. These mold types are used exclusively for the fabrication of dissolvable microneedles and are not applicable to the manufacture of solid, hollow, or coated microneedle systems, which require distinct fabrication processes and materials.

The drying step may be performed under a range of conditions, depending on the characteristics of the polymer formulation, the presence and stability of active ingredients, and the intended commercial application. In various embodiments, drying temperatures may range from approximately 4° C. to 60° C., with preferred drying occurring between 20° C. and 40° C. to accommodate temperature-sensitive compounds. Drying times may vary from 30 minutes to 72 hours, depending on ambient humidity, formulation viscosity, mold material, and microneedle geometry. For example, high-viscosity formulations may require extended drying times to achieve full solvent evaporation and structural rigidity, while low-viscosity formulations may be rapidly dried using centrifugal or vacuum-assisted methods.

When vacuum drying is employed, the pressure may range from approximately 0.1 mbar to 100 mbar, with a preferred range between 1 mbar and 10 mbar to balance drying speed and product stability. In freeze-drying processes, freezing temperatures may range from−-80° C. to −20° C. , with primary drying carried out under pressures below 0.5 mbar and secondary drying completed at elevated temperatures to ensure residual moisture removal. The total freeze-drying process may range from 6 hours to 72 hours, depending on batch size and formulation sensitivity. Centrifugal drying may be performed at rotational speeds ranging from approximately 500 to 10,000 revolutions per minute (rpm), with spin durations ranging from 30 seconds to 30 minutes, depending on the thickness of the formulation and the mold design.

In certain embodiments, multi-step drying processes may be used to optimize both structural strength and active compound retention. For example, a low-temperature air drying phase may be followed by a brief vacuum phase to eliminate trapped moisture and minimize porosity. Additionally, formulations intended for layered microneedle structures (e.g., tip-loaded systems or biphasic drug release systems) may be subjected to sequential drying cycles, with partial drying after the first layer is cast to prevent interlayer mixing, followed by final drying once the full microneedle structure is assembled.

Post-drying, the microneedle arrays may be further stabilized by conditioning under controlled temperature and humidity, typically within the range of 15° C. to 30° C. and 15% to 45% relative humidity, for a period of 12 to 72 hours to ensure consistent mechanical performance and long-term storage stability. Packaging under low-moisture barrier conditions or inert atmosphere may also be employed to preserve structural integrity and prevent premature degradation, particularly for moisture-sensitive APIs or food-grade formulations.

In certain embodiments, the dried dissolvable microneedle system may be subjected to a conditioning step under controlled temperature and humidity conditions to improve mechanical stability, moisture content, and dissolution reproducibility. The conditioning process may be performed at a temperature ranging from approximately 10° C. to 40° C. and a relative humidity between 10% and 60%, for a period of 6 hours to 96 hours. These broader conditions may be selected based on the material composition of the microneedles, the sensitivity of the encapsulated bioactive compound, or storage stability requirements.

These drying range, conditions, and combinations are not limited to the examples disclosed herein. Any process capable of producing a solidified microneedle array with mechanical integrity sufficient for skin insertion, while preserving the bioactivity, taste, texture, or efficacy of the encapsulated agent whether pharmaceutical, nutraceutical, cosmetic, or food-grade may be employed. The invention encompasses all such drying methods, parameters, and conditions, including those not yet commercially developed or identified at the time of this application, provided they achieve the functional requirements of dissolvable microneedle fabrication and performance.

In certain embodiments, the dissolvable microneedle array may be mounted on a support layer that includes an adhesive component. This adhesive may be a medical-grade pressure-sensitive adhesive, hydrogel adhesive, or other biocompatible formulation designed to facilitate temporary adhesion to the skin during application. In other embodiments, no additional adhesive is required, as the hydrophilic polymer matrix used to form the microneedles, such as hyaluronic acid, pullulan, or carboxymethylcellulose, may become inherently tacky upon contact with skin moisture, thereby providing sufficient adherence for the duration of the application. In such cases, the microneedle array may be applied directly to the skin and manually pressed to ensure insertion, without the need for an external adhesive. The use or omission of an adhesive component may be selected based on the intended therapeutic application, wear time, skin location, or user preference. The invention is not limited to the use of an adhesive, and any configuration that provides stable skin contact during microneedle insertion and dissolution may be employed.

### Uses of DMN System

The present invention relates to the use of dissolvable microneedles for a broad range of applications.

DMN patches are helpful for transdermal delivery of nutrients that are too large to diffuse through the skin in a traditional skin patch, such as proteins. Dissolving microneedles can be used for the transdermal delivery of high molecular weight compounds like proteins, peptides, and vaccines, as they can create microchannels in the skin to facilitate drug absorption.

One use for a DMN patch is to deliver solid lipid nanoparticles (SLNs) directly into the skin. SLNs are submicron-sized particles composed of biocompatible lipids, designed to encapsulate and protect bioactive compounds while enhancing their bioavailability and stability. SLNs exhibit several advantages for delivering at least one bioactive compound, including their small size, controlled release properties, and ability to protect sensitive compounds from degradation caused by environmental factors such as light, heat, or oxidation. SLNs can be used for encapsulating compounds derived from Spirulina, as described above, specifically C-PC, which has numerous uses in the treatment of a wide array of medical conditions.

The incorporation of Spirulina into dissolvable microneedles (DMNs) enables a novel transdermal delivery platform that expands the therapeutic and preventive applications of this nutrient-dense microalga. By bypassing the gastrointestinal tract and delivering Spirulina's bioactive compounds directly through the skin, DMNs enhance bioavailability, stability, and patient compliance. Spirulina DMNs may be used for immune support by modulating immune cell activity and enhancing host defense without overstimulation. Their potent anti-inflammatory properties make them suitable for managing chronic inflammatory conditions such as arthritis, fibromyalgia, and autoimmune flares. In neuroprotective applications, Spirulina DMNs may help reduce oxidative damage to neurons and support cognitive clarity and mitochondrial function. They can also be used for systemic antioxidant support and detoxification by neutralizing free radicals and promoting glutathione production.

Additionally, Spirulina DMNs offer a non-oral route for delivering bioavailable iron and other nutrients to individuals with anemia or nutrient malabsorption, particularly in pediatric and geriatric populations. In metabolic and cardiovascular applications, these microneedles can help regulate blood sugar levels, improve insulin sensitivity, and support healthy lipid profiles by lowering LDL cholesterol and blood pressure while increasing HDL levels. In dermatologic use, Spirulina DMNs may deliver antioxidants and anti-aging compounds directly to the skin to improve hydration, reduce oxidative stress, and promote collagen production. For cancer patients, Spirulina DMNs may serve as supportive therapy by reducing treatment-induced oxidative stress and inflammation.

Veterinary applications include delivering immune and anti-inflammatory support to companion animals through patches applied to the ear or skin, avoiding oral or injectable dosing. Oral health formulations may involve buccal or gumline DMNs containing Spirulina to manage gingival inflammation and support mucosal healing. Furthermore, Spirulina DMNs can be formulated into daily wellness or energy-boosting patches for busy individuals, offering a stimulant-free, nutrient-rich solution to support vitality and mental clarity.

Finally, Spirulina may be co-formulated with other bioactive agents, such as vitamin D3, magnesium, or curcumin, in a single DMN patch for synergistic effects across multiple health domains. These applications demonstrate the unique potential of Spirulina-embedded DMNs for safe, effective, and targeted delivery of natural therapeutic compounds across a wide range of use cases.

Active components of Spirulina, C-Phycocyanin (C-PC), a blue pigment-protein complex found in Spirulina, has been studied for its potent biological activities and therapeutic potential across a range of health conditions. C-PC exhibits strong antioxidant properties by scavenging free radicals and inhibiting lipid peroxidation, thereby protecting cells and tissues from oxidative stress. It has also been shown to have significant anti-inflammatory effects, primarily through downregulation of pro-inflammatory cytokines such as TNF-α, IL-6, and IL-1β, and by modulating the NF-κB and COX-2 pathways. These mechanisms make C-PC a promising candidate for managing conditions such as arthritis, autoimmune diseases, and inflammatory skin disorders. Furthermore, C-PC demonstrates neuroprotective activity by inhibiting neuroinflammation and oxidative damage to neuronal cells, with studies indicating potential benefits in cognitive health and the prevention of neurodegenerative diseases such as Alzheimer's and Parkinson's.

In addition to its anti-inflammatory and neuroprotective effects, C-PC has been shown to exert anticancer activity through several mechanisms, including the induction of apoptosis in tumor cells, inhibition of tumor growth, and suppression of angiogenesis. In vitro and in vivo studies have demonstrated its efficacy in liver, colon, and breast cancer models. C-PC also supports immune modulation, enhancing the function of macrophages, lymphocytes, and natural killer (NK) cells, making it suitable for immunocompromised individuals or as a complement to vaccination strategies. Its analgesic potential has been demonstrated through COX-2 inhibition, suggesting a role as a natural, non-opioid alternative for pain management. Emerging research further supports its role in hepatoprotection, helping to reduce liver enzyme levels and prevent chemically induced liver damage, and in cardiovascular health, by reducing blood pressure and improving endothelial function through nitric oxide modulation.

Preclinical evidence also indicates radioprotective properties of C-PC, with the ability to mitigate DNA damage and oxidative injury caused by ionizing radiation, highlighting its potential as a supportive therapy during cancer treatment. Additional studies suggest C-PC may have antiviral activity, likely linked to its immunostimulatory and antioxidant functions, though mechanisms are still being explored. These multifunctional bioactivities position C-PC as a highly valuable natural therapeutic compound for applications in transdermal delivery systems, particularly dissolvable microneedles, where its instability under heat, pH variation, and enzymatic degradation can be mitigated by protective encapsulation. The delivery of C-PC via DMNs offers a novel, non-invasive method to harness its therapeutic effects across systemic and localized conditions, including inflammation, oxidative stress, immune dysfunction, neurological disorders, cancer, pain, liver disease, and metabolic syndromes.

The present invention also encompasses a broad range of novel therapeutic and preventive applications of a C-Phycocyanin (C-PC) dissolvable microneedle (DMN) system. C-PC, a potent antioxidant and anti-inflammatory protein derived from Spirulina, can be delivered transdermally through DMNs to bypass gastrointestinal degradation and improve bioavailability for local and systemic action. Applications of the C-PC DMN system include chronic and acute anti-inflammatory therapy, such as for arthritis, fibromyalgia, lupus, post-injury inflammation, and skin conditions like atopic dermatitis and eczema. Neuroprotective applications include cognitive performance support, neuroinflammation reduction in conditions such as long COVID and chronic fatigue syndrome, and adjunctive therapy for neurodegenerative diseases such as Alzheimer's and Parkinson's. C-PC DMNs may also serve as a non-opioid alternative for pain management, including localized musculoskeletal pain, dental pain via buccal application, and chronic pain conditions such as neuropathy or fibromyalgia.

In addition, C-PC DMNs may be employed for systemic antioxidant therapy, including oxidative stress reduction, mitochondrial support, and performance recovery in high-demand mental or physical settings. Immune modulation applications include support for autoimmune conditions, immune enhancement in aging or immunocompromised individuals, and as an adjunct to vaccines or immunotherapy. In oncology support, C-PC DMNs may be used to protect healthy tissues during chemotherapy or radiation, reduce treatment-associated oxidative stress, and support recovery in post-treatment phases, as well as provide immunomodulatory and antiangiogenic effects in tumor microenvironments. The invention further includes radioprotective applications, such as mitigation of radiation damage in cancer patients or occupational exposures, and as a protective patch for emergency radiation or chemical exposure scenarios.

C-PC DMNs may also be applied in hepatoprotective therapy, offering protection against drug-induced liver injury, viral hepatitis, and metabolic liver conditions such as fatty liver disease. Cardiometabolic applications include blood pressure regulation, endothelial protection, and reduction of metabolic inflammation in insulin-resistant or obese individuals. In the oral health domain, C-PC DMNs may be applied to the gumline or buccal mucosa to treat gingivitis, periodontitis, and oral ulcers, and serve as an adjunct therapy for oral leukoplakia or TMJ-related inflammation. The invention further supports pediatric and geriatric applications by offering a safe, non-oral method for delivering antioxidant and immune-enhancing compounds in populations with swallowing difficulties or malabsorption. Skin health applications include transdermal antioxidant delivery for anti-aging, hyperpigmentation reduction, support for inflammatory skin conditions such as psoriasis and rosacea, and wound healing enhancement.

C-PC DMNs may also be used to support mood stability, reduce neuroinflammation associated with anxiety or depression, and serve as adjunctive therapy for reducing side effects of psychotropic medications. Performance and recovery use cases include patches for physical and cognitive recovery in athletes and professionals, jet lag recovery, and inflammation-reducing sleep support. The invention further encompasses multi-pathway therapeutic combinations, such as C-PC co-formulated with magnesium, vitamin D, curcumin, resveratrol, or natural nootropics like L-theanine or bacopa for synergistic outcomes. Dual-layer DMN systems are also contemplated, wherein C-PC is combined with immune-enhancing or skin barrier-supportive agents in a staged release format.

Veterinary applications of C-PC DMNs include non-oral delivery for companion animals to manage inflammation, pain, or immune dysfunction, especially in aging, post-surgical, or immunosuppressed animals. To the best of current knowledge, several of these applications represent novel uses not disclosed in prior art, including but not limited to: transdermal use of C-PC via DMNs for neuroinflammation and brain fog; radioprotective use of C-PC DMNs in non-cancer scenarios such as space travel or nuclear exposure; buccal and gumline application of C-PC DMNs for oral inflammatory and precancerous lesions; use of C-PC DMNs in the management of post-viral syndromes such as long COVID; co-formulated DMNs combining C-PC with mitochondrial cofactors, nootropics, or sleep recovery agents; and veterinary applications of C-PC DMNs for non-invasive nutrient or therapeutic delivery to companion animals. These applications underscore the versatility and innovation of the proposed C-PC DMN delivery platform across human and animal health.

Spirulina-embedded DMN systems can also be used for the treatment of Bell's Palsy, a neurological condition characterized by acute, unilateral facial paralysis due to inflammation or viral reactivation affecting the facial nerve. Spirulina contains a rich array of bioactive compounds, including phycobiliproteins, superoxide dismutase, beta-carotene, and sulfated polysaccharides, which collectively exhibit potent anti-inflammatory, antioxidant, neuroprotective, and antiviral properties. These properties make Spirulina particularly well-suited for addressing the multifactorial pathophysiology of Bell's Palsy. Transdermal delivery via dissolvable microneedles enables targeted, localized release of Spirulina's therapeutic compounds near the preauricular or facial nerve region, bypassing gastrointestinal degradation and first-pass metabolism. This delivery system allows for sustained anti-inflammatory and neuroprotective effects, while the antiviral components may suppress underlying herpes simplex virus activity, a suspected trigger in many Bell's Palsy cases. The system provides a non-invasive, painless, and self-administered alternative to conventional corticosteroid or antiviral therapies, with the added benefit of nutritional and immunomodulatory support. To the best of current knowledge, the use of Spirulina in a dissolvable microneedle system for the treatment of Bell's Palsy has not been previously disclosed in the prior art, representing a novel therapeutic application of this natural compound in a precision delivery format.

Spirulina is a complex mixture of bioactives, including hydrophilic (e.g., phycocyanin) and lipophilic components, making Spirulina more challenging than other materials (e.g., beta-carotene) to encapsulate in solid lipid nanoparticles (SLNs). For example, encapsulation of beta-carotene primarily focuses on oxidation prevention and achieving uniformity, often resulting in smaller particle sizes and lower polydispersity index (PDI) due to its simpler composition. In contrast, Spirulina requires dual-phase stabilization strategies to accommodate its diverse compounds, leading to more complex processing methods and a need for customized stabilizers to ensure particle uniformity and stability. Additionally, Spirulina bioactives have higher susceptibility to degradation under environmental stress (light, pH, and temperature), requiring stabilization approaches that are unnecessary for single-compound formulations like beta-carotene SLNs.

Spirulina (Arthrospira platensis) is a free-floating, filamentous cyanobacteria that is widely used as a dietary supplement. Spirulina is a nutrient-dense blue-green microalga known for its wide range of health benefits, primarily due to its high concentrations of complete protein, 9 essential amino acids, vitamins, minerals, and antioxidants. It is particularly rich in B vitamins, iron, magnesium, potassium, and trace minerals such as selenium and zinc, making it a powerful plant-based supplement that supports overall health. Spirulina contributes to cardiovascular health by lowering LDL cholesterol and blood pressure while increasing HDL cholesterol. It may help regulate blood sugar levels and improve insulin sensitivity, making it beneficial for individuals with metabolic concerns. Spirulina also supports red blood cell production and may help reduce anemia, especially in individuals with iron deficiency. Additionally, it plays a role in detoxification by binding to heavy metals and supporting liver function. Other reported benefits include improved cognitive clarity, gut microbiome balance, skin health, and enhanced exercise recovery and endurance.

Spirulina exhibits a wide range of therapeutic properties beyond its nutritional value, including notable anti-inflammatory, antiviral, and anticancer effects. Its anti-inflammatory activity is supported by its ability to inhibit pro-inflammatory cytokines such as TNF-α, IL-6, and IL-1β, while modulating key inflammatory pathways like NF-κB and COX-2. These actions contribute to the reduction of systemic inflammation and support immune regulation. Spirulina also demonstrates broad-spectrum antiviral effects, primarily due to the presence of sulfated polysaccharides such as calcium spirulan, which interfere with viral attachment and replication. These antiviral properties have been observed against various enveloped viruses, including herpes simplex virus, influenza A, cytomegalovirus, and HIV. In addition, Spirulina has shown anticancer potential in both in vitro and animal studies, where it has been observed to suppress tumor growth, induce apoptosis, and reduce DNA damage associated with carcinogenesis. Clinical studies have also suggested its ability to reverse precancerous oral lesions and enhance natural killer cell activity, indicating its potential role in cancer prevention and immune surveillance.

Beyond these core therapeutic benefits, emerging research has revealed other promising applications of Spirulina. It has demonstrated radioprotective effects by reducing oxidative damage caused by radiation exposure, making it a potential adjunct in cancer radiotherapy or environmental protection. Spirulina also appears to have antihistamine-like effects by reducing histamine release, which may help alleviate symptoms of allergic rhinitis and other histamine-mediated conditions. In liver health, Spirulina has been shown to protect against fatty liver, hepatic fibrosis, and drug-induced liver injury by improving liver enzyme profiles and reducing oxidative stress. Additionally, its rich content of zeaxanthin and beta-carotene supports eye health and may help prevent macular degeneration and cataracts. Spirulina has also been linked to weight management benefits by promoting satiety, improving lipid metabolism, and reducing markers of metabolic dysfunction. Finally, animal studies have indicated potential benefits for male fertility, including improved sperm quality and testosterone levels. Collectively, these findings position Spirulina as a multifaceted bioactive compound with significant potential in preventive health, functional nutrition, and therapeutic applications.

The application of DMN containing C-Phycocyanin (C-PC), a key bioactive compound of Spirulina, can also be used for the treatment of Bell's Palsy. C-PC possesses potent anti-inflammatory and neuroprotective properties that make it especially well-suited for managing this condition. It has been shown to inhibit pro-inflammatory cytokines and downregulate the NF-κB and COX-2 pathways, thereby reducing localized nerve inflammation. Additionally, C-PC provides antioxidant and mitochondrial support that may protect facial nerve cells from oxidative damage and promote functional nerve recovery. Given that herpes simplex virus is a suspected etiological factor in Bell's Palsy, the antiviral properties of C-PC further enhance its therapeutic potential. The use of DMNs allows for non-invasive, localized delivery of C-PC directly to the affected facial area, such as the preauricular or cheek region, bypassing the gastrointestinal tract and avoiding systemic side effects associated with oral corticosteroids. This method offers a needle-free, pain-free approach for targeted therapy, which may be self-administered and tailored for once- or twice-daily application over a defined treatment period. To the best of current knowledge, the use of C-PC in a DMN system for the treatment of Bell's Palsy has not been previously disclosed, thereby representing a novel and clinically significant advancement in transdermal neurotherapeutic delivery.

However, C-PC is a protein and inherently unstable, particularly when exposed to heat, light, pH changes, or digestive enzymes, which significantly limits its bioavailability and therapeutic potential when administered in traditional oral or topical forms. To overcome this limitation, it is desirable to encapsulate C-PC into a carrier, such as SLNs, electrospun nanofibers, bacterial cellulose-based hydrogels, biodegradable microneedle matrices, silk fibroin microparticles, ferrofluid-assisted lipid vesicles, functionalized marine polysaccharide complexes (e.g., fucoidan, ulvan, or laminarin-derived carriers) or equivalent carrier that is currently available or not yet developed to protect C-PC from degradation while enabling targeted or sustained delivery for oral, transdermal, or mucosal administration.

C-Phycocyanin (C-PC) is a potent anti-inflammatory natural protein by itself. Phycocyanin is an FDA-approved water-soluble, non-toxic, and blue-colored photosynthetic pigment, which has undergone rigorous safety evaluations. In various color additive petitions, FDA has determined an Acceptable Daily Intake (ADI) of 1.0-1.8 g/p/d, for phycocyanin, and a No Observed Effect Level (NOEL) of 108-184.5 g/p/d in a GRAS submission for phycocyanin. It has been used in food, cosmetics, and pharmaceutical industries. C-PC, a major active compound in Spirulina, has also been widely used as a nutritional supplement. The anti-inflammatory function of C-PC has been extensively investigated. For example, oral administration of C-PC significantly reduced intestine inflammation and colitis in animal models. C-PC also showed promising efficacy in modulating organ inflammation beyond the digestive system. In animal models, C-PC, delivered via nasal spray or oral administration, showed the potential to reduce inflammation in diseases of the liver, lungs, and brain. Although the molecular mechanism of C-PC's anti-inflammatory function is not fully understood, previous studies suggested that C-PC promotes the relative expression of antioxidant enzymes and down-regulates the TLR2/MyD88/NF-κB pathway, leading to anti-inflammatory roles. Other studies suggested that C-PC activates the Nrf2 signaling pathway by upregulating the content of Nrf2 and HO-1 in various tissues, thus reducing inflammation. The other mechanism is through the inhibition of NADPH oxidase mimicking the function of biliverdin.

Although limited clinical studies on its direct effect exist, several Spirulina trials support its anti-inflammatory function. In a clinical trial for chronic periodontitis, Spirulina gel applied with the Scaling and Root Planing (SRP) treatment significantly improved the clinical outcome, likely associated with the anti-inflammatory activity of C-PC in Spirulina. Another clinical trial in patients with oral submucous fibrosis (OSMF) reported that daily oral administration of Spirulina significantly reduced the mouth stiffness, oral ulcers, and erosions associated with the disease. Spirulina also showed a promising functionality as adjuvant chemotherapy to improve the immune function of tumor patients by significantly increasing IgM levels and CD8+T cell numbers29. As C-PC is one of the major bioactive ingredients in Spirulina, these clinical data support its therapeutic potential as an anti-inflammatory natural compound. Supported by the promising clinical outcomes, there is an ongoing clinical trial evaluating the efficacy of C-PC against the neurotoxicity of oxaliplatin-based chemotherapy in patients with gastrointestinal cancer.

A DMN patch embedded with C-PC may be used in patients with inflammatory bowel disease (IBD). Crohn's Disease is a type of inflammatory bowel disease (IBD) that causes inflammation in the digestive tract, which may lead to symptoms of diarrhea, abdominal pain, nausea, fever, loss of appetite, weight loss, and fatigue. It affects over 1 million people in the US and 2.3 million globally. The disease substantially burdens healthcare systems and diminishes people's quality of life. Crohn's Disease causes chronic gastrointestinal inflammation, which can lead to severe complications, such as strictures, fistulas, and abscesses, which often require surgery or hospitalization. Current Crohn's Disease treatments, including oral immunosuppressants, intravenous or subcutaneous biologics, and surgery, have significant side effects and high costs.

While C-PC is helpful for resolving Crohn's Disease related inflammation, there are two problems that have thus limited its use. C-PC is unstable-it is sensitive to pH, temperature, and light, so it can be very easily inactivated during storage. C-PC molecules are large, making transdermal application impractical. Moreover, oral administration of C-PC may not be the best route of administration for treating Crohn's Disease because patients experience gut inflammation, which reduces the absorption of nutrients from the small intestine.

SLNs can solve both the problem of preserving the bioactivity of C-PC during storage and the problem of decreased gastrointestinal absorption. Since the C-PC is encapsulated within a lipid membrane, it is protected from degradation, making it easier to use it in pharmaceuticals. Furthermore, it is possible to deliver SLNs transdermally using a DMN patch, bypassing the digestive system altogether.

There are many other bioactive compounds that can be encapsulated in SLNs and delivered transdermally using a DMN patch. For example, since many elderly people are deficient in vitamin D and also have trouble absorbing it orally, it is desirable to deliver vitamin D transdermally; however, traditional transdermal patches have shown limited efficacy in delivering vitamin D. Using an SLN to encapsulate vitamin D enhances bioavailability and protects the encapsulated material from degradation caused by light, heat, or oxidation. The SLNs provide a gradual release of active ingredients, ensuring a consistent supply of vitamin D over time and reducing the need for frequent dosing. Using a dissolvable microneedle (DMN) patch to deliver the SLNs will ensure that the active ingredients are delivered directly into the dermis, enabling faster and more efficient absorption compared to the passive diffusion mechanism of traditional patches.

The delivery of amino acids and/or peptides can be used for a range of applications. DMNs provide significant advantages in bioavailability, stability, ease of administration, and avoidance of gastrointestinal or first-pass hepatic metabolism. The invention is suitable for use in humans, animals, and plants, and may address unmet needs across nutritional supplementation, therapeutic intervention, cosmetic enhancement, and biological stimulation.

In human applications, the invention may be used for the delivery of essential, non-essential, or conditionally essential amino acids, as well as functional peptides, to support therapeutic, preventive, or cosmetic outcomes. In some embodiments, amino acids embedded into DMNs may be used to treat and manage malnutrition, sarcopenia, cachexia, Crohn's disease, metabolic disorders, wound healing, immune modulation, neurodegenerative diseases, cardiovascular support, and endocrine disorders. The invention may further be used for delivery of bioactive peptides in DMNs and may include immune support, stress adaptation, athletic performance, muscle recovery, and mood regulation. In cosmetic and dermatological applications, the invention may be used to deliver amino acids, collagen-stimulating peptides, anti-wrinkle or anti-pigmentation agents, hydrating peptides, antimicrobial peptides, and compounds that promote hair growth or skin barrier repair.

In veterinary applications, the invention may be applied to livestock, companion animals, and exotic species for nutritional supplementation, post-operative healing, growth promotion, stress reduction, and immune system modulation. The system may also serve as a platform for delivering peptide-based medications or vaccines in a painless, minimally invasive manner. In performance and production animals, such as horses, cattle, poultry, or aquaculture species, the invention may enhance muscle mass, feed efficiency, recovery from disease, or overall vitality.

In agricultural applications, the invention may be adapted to deliver amino acids or peptides to plants for biostimulant or biofertilizer purposes. Specific applications include stimulation of plant growth, enhancement of root development, promotion of drought or salinity tolerance, improvement of nutrient uptake, or activation of plant immune defenses. Amino acid and peptide delivery may also enhance microbial health in the rhizosphere or serve as an environmentally friendly alternative to synthetic agrochemicals. In certain embodiments, the delivery platform may be modified for foliar application, root zone delivery, or integration into hydrogel matrices or biodegradable films for controlled release in plant systems.

Collectively, the invention enables the delivery of stabilized amino acids and peptides through dissolvable microneedles or analogous delivery systems for a wide range of biologically relevant applications in healthcare, wellness, veterinary medicine, and sustainable agriculture.

The present invention includes the transdermal or intradermal delivery of amino acids and peptides derived from algae, embedded in dissolvable microneedles for use in therapeutic, cosmetic, nutraceutical, or wellness applications. The algae may include, but are not limited to, microalgae, macroalgae, cyanobacteria, red algae (Rhodophyta), brown algae (Phaeophyceae), and green algae (Chlorophyta), including both marine and freshwater species. This encompasses Spirulina platensis, Laminaria japonica (kelp), Sargassum species, Chlorella vulgaris, Porphyra yezoensis, Dunaliella salina, Ecklonia cava, and any other known, cultivated, wild-type, genetically modified, or as-yet-undiscovered algal strains capable of producing amino acids or peptides with biological activity.

The amino acids and peptides may be isolated through enzymatic hydrolysis, aqueous extraction, fermentation, or other processing methods and may include essential amino acids, non-essential amino acids, conditionally essential amino acids, protein hydrolysates, short-chain peptides, or bioactive peptide fragments. These compounds may exhibit functions such as collagen stimulation, skin hydration, antioxidant activity, immune modulation, muscle recovery, wound healing, pigmentation support, or hair growth enhancement. Examples of amino acids that may be derived from algal sources include leucine, isoleucine, valine, arginine, lysine, tryptophan, tyrosine, cysteine, glutamic acid, aspartic acid, and others. However, these examples are not intended to be limiting.

In some embodiments, the invention broadly encompasses any amino acid, peptide, or functional derivatives that can be derived or isolated from algae, whether currently characterized or discovered in the future. These compounds may be incorporated directly into the microneedle matrix or pre-encapsulated using nanocarriers such as solid lipid nanoparticles, liposomes, or polymeric particles to improve stability, bioavailability, or controlled release. The delivery of algae-derived amino acids and peptides via dissolvable microneedles provides a minimally invasive, needle-free route of administration that bypasses gastrointestinal degradation and enables localized or systemic effects, offering a novel and sustainable platform for enhancing human and animal health, as well as plant and skin wellness.

In addition to amino acids and peptides, dissolvable microneedle systems or analogous delivery platforms may be utilized to deliver essential and trace minerals to plants. These minerals may promote nutrient balance, improve photosynthesis, enhance enzymatic activity, and support structural development in plants. The DMN formulations may be adapted for foliar application, soil-embedded use, or biodegradable matrix integration for controlled nutrient release. Mineral-enriched DMNs may also enhance rhizosphere health and provide a sustainable, targeted alternative to conventional synthetic fertilizers, particularly in agriculture and organic farming systems.

Other vitamins and minerals may also be embedded into a DMN system for transdermal delivery. While vitamin D is a small molecule (~385 Da), utilizing a nanocarrier, such as a SLN, can help increase the stability and controlled release of the vitamin. Additionally, encapsulating vitamins into a carrier may enable the absorption of larger micronutrients such as Vitamin B12 (1,355 Da) and proteins that would otherwise not be able to be absorbed through the skin due to their larger molecular sizes. Vitamin B12, which is essential for nerve function and red blood cell production, is particularly beneficial for individuals with absorption challenges. Iron, particularly non-heme iron, is often poorly absorbed in conventional oral formulations. SLN encapsulation significantly improves its bioavailability and reduces oxidation, ensuring better absorption. Magnesium, important for muscle function, relaxation, and skin hydration, benefits from SLN encapsulation, improving its solubility and bioavailability. Zinc is vital for immune health, wound healing, and skin repair. Folate (Vitamin B9) plays a role in prenatal health and cardiovascular function. Coenzyme Q10, a powerful antioxidant, is beneficial for cardiovascular health and skin rejuvenation. Copper and selenium, trace minerals, are crucial for enzymatic functions and provide antioxidant protection. Manganese supports connective tissue and bone health, while chromium enhances glucose metabolism.

The present invention further includes the use of algae-based compounds and materials embedded within dissolvable microneedle (DMN) systems for the treatment, prevention, or general wellness support related to diabetic foot ulcers (DFUs) and broader diabetic care. These compounds may include, but are not limited to, phycobiliproteins such as C-Phycocyanin, marine-derived carotenoids such as fucoxanthin and astaxanthin, sulfated polysaccharides including fucoidan, ulvan, and laminarin, polyphenols such as dieckol or phlorotannins, chlorophyll derivatives, algal amino acids, collagen-like peptides, and fermented or enzymatically processed microalgae extracts containing trace minerals and regenerative cofactors. These compounds may be used alone or in combination with other bioactive agents including peptides, antioxidants, trace elements, vitamins, plant extracts, probiotics, or synthetic therapeutics to reduce inflammation, promote angiogenesis, enhance tissue regeneration, support nerve repair, or manage microbial burden in ulcer-prone skin.

Such algae-based compounds and additional bioactives may be bioengineered, structurally modified, or combined with delivery-enhancing technologies to improve their performance in diabetic wound care. This may include microencapsulation, nanoencapsulation, or liposomal delivery to protect sensitive compounds from degradation and to enable controlled or time-dependent release. For example, C-Phycocyanin may be nanoencapsulated in lipid-based carriers to enhance thermal and pH stability, or sulfated polysaccharides may be crosslinked with biocompatible polymers to prolong residence time in the skin. Stabilizers such as trehalose, glycerol, or plant-derived gums may also be included to preserve bioactivity during storage and dissolution. Targeted delivery features, including pH-sensitive release, enzyme-triggered release, or surface functionalization for tissue targeting, may be incorporated to increase efficacy in ulcer microenvironments characterized by oxidative stress, poor perfusion, and microbial presence.

Algae-derived biopolymers such as alginate, carrageenan, or agarose may also serve as structural components or carriers in the microneedle matrix, supporting hydration, adhesion, and biocompatibility. The microneedle system may be designed for therapeutic use on active ulcers, prophylactic use in high-risk areas, or general maintenance of skin integrity and circulation in diabetic individuals. While specific examples of algae-derived compounds and delivery strategies are provided herein, the invention is not limited to these alone, and includes all naturally occurring, synthetic, recombinant, or as-yet-undiscovered algal compounds and combinations thereof with equivalent or enhanced functionality. Furthermore, this invention encompasses future innovations in delivery technology or compound formulation that improve safety, efficacy, stability, or clinical applicability in diabetic foot ulcer management and diabetic health more broadly.

The delivery of amino acids and/or peptides through dissolvable microneedles provides significant advantages in bioavailability, stability, ease of administration, and avoidance of gastrointestinal or first-pass hepatic metabolism. The invention is suitable for use in humans, animals, and plants, and may address unmet needs across nutritional supplementation, therapeutic intervention, cosmetic enhancement, and biological stimulation.

In human applications, the invention may be used for the delivery of essential, non-essential, or conditionally essential amino acids, as well as functional peptides, to support therapeutic, preventive, or cosmetic outcomes. In some embodiments, medical applications can be used to treat or manage malnutrition, sarcopenia, cachexia, Crohn's disease, metabolic disorders, wound healing, immune modulation, neurodegenerative diseases, cardiovascular support, and endocrine disorders. The invention may further be used to deliver bioactive peptides such as CGRP antagonists, oxytocin analogs, melanocyte-stimulating hormone (MSH) analogs, neuropeptide Y, and Substance P. DMNs embedded with amino acids or peptides may be used for preventive health conditions such as immune support, stress adaptation, athletic performance, muscle recovery, and mood regulation. In cosmetic and dermatological applications, the invention may be used to deliver collagen-stimulating peptides, anti-wrinkle or anti-pigmentation agents, hydrating peptides, antimicrobial peptides, and compounds that promote hair growth or skin barrier repair.

In veterinary applications, the invention may be applied to livestock, companion animals, and exotic species for nutritional supplementation, post-operative healing, growth promotion, stress reduction, and immune system modulation. The system may also serve as a platform for delivering peptide-based medications or vaccines in a painless, minimally invasive manner. In performance and production animals, such as horses, cattle, poultry, or aquaculture species, the invention may enhance muscle mass, feed efficiency, recovery from disease, or overall vitality.

In agricultural applications, the invention may be adapted to deliver amino acids or peptides to plants for biostimulant or biofertilizer purposes. Specific applications include stimulation of plant growth, enhancement of root development, promotion of drought or salinity tolerance, improvement of nutrient uptake, or activation of plant immune defenses. Amino acid and peptide delivery may also enhance microbial health in the rhizosphere or serve as an environmentally friendly alternative to synthetic agrochemicals. In certain embodiments, the delivery platform may be modified for foliar application, root zone delivery, or integration into hydrogel matrices or biodegradable films for controlled release in plant systems.

Collectively, the invention enables the delivery of stabilized amino acids and peptides through dissolvable microneedles or analogous delivery systems for a wide range of biologically relevant applications in healthcare, wellness, veterinary medicine, and sustainable agriculture.

The present invention includes the transdermal or intradermal delivery of amino acids and peptides derived from algae, embedded in dissolvable microneedles for use in therapeutic, cosmetic, nutraceutical, or wellness applications. In some embodiments, the algae may include microalgae, macroalgae, cyanobacteria, red algae (Rhodophyta), brown algae (Phaeophyceae), and green algae (Chlorophyta), including both marine and freshwater species. This encompasses Spirulina platensis, Laminaria japonica (kelp), Sargassum species, Chlorella vulgaris, Porphyra yezoensis, Dunaliella salina, Ecklonia cava, and any other known, cultivated, wild-type, genetically modified, or as-yet-undiscovered algal strains capable of producing amino acids or peptides with biological activity.

The amino acids and peptides may be isolated through enzymatic hydrolysis, aqueous extraction, fermentation, or other equivalent processing method and may include essential amino acids, non-essential amino acids, conditionally essential amino acids, protein hydrolysates, short-chain peptides, or bioactive peptide fragments. These compounds may exhibit functions such as collagen stimulation, skin hydration, antioxidant activity, immune modulation, muscle recovery, wound healing, pigmentation support, or hair growth enhancement. Examples of amino acids that may be derived from algal sources include leucine, isoleucine, valine, arginine, lysine, tryptophan, tyrosine, cysteine, glutamic acid, aspartic acid, and others. However, these examples are not intended to be limiting in amino acid or human application

The invention broadly encompasses any amino acid or peptide that can be derived or isolated from algae, whether currently characterized or discovered in the future. These compounds may be incorporated directly into the microneedle matrix or pre-encapsulated using nanocarriers such as solid lipid nanoparticles, liposomes, or polymeric particles to improve stability, bioavailability, or controlled release. The delivery of algae-derived amino acids and peptides via dissolvable microneedles provides a minimally invasive, needle-free route of administration that bypasses gastrointestinal degradation and enables localized or systemic effects, offering a novel and sustainable platform for enhancing human and animal health, as well as plant and skin wellness.

The present invention further encompasses a dissolvable microneedle (DMN) system for the treatment, prevention, or management of sciatica and related nerve inflammation, wherein the pathology may be associated with viral, inflammatory, or idiopathic causes. In one embodiment, the DMN system is designed for transdermal delivery of one or more antiviral, anti-inflammatory, neuroprotective, or immunomodulatory agents, delivered locally or systemically through the skin to support sciatic nerve recovery. The microneedles may be applied to anatomical regions overlying or adjacent to the path of the sciatic nerve, such as the lower lumbar spine, sacral area, gluteal region, or posterior thigh. The formulation may include known or novel antiviral agents selected from natural, synthetic, or bioengineered compounds, such as zinc salts, lysine, lemon balm extract, monolaurin, licorice root extract (glycyrrhizin), olive leaf extract, cat's claw, shilajit derivatives, or algae-derived sulfated polysaccharides, including calcium spirulan, ulvan, fucoidan, or other marine bioactives with known or potential antiviral activity.

These compounds may be used alone or in combination with other bioactive agents that exhibit synergistic or supportive properties, including anti-inflammatory compounds (e.g., curcumin, resveratrol, C-Phycocyanin), nerve-supportive nutrients (e.g., methylcobalamin, magnesium, alpha-lipoic acid), adaptogens (e.g., ashwagandha, Rhodiola), or mitochondrial co-factors (e.g., coenzyme Q10, NAD+precursors). The microneedle matrix may be composed of biodegradable, biocompatible materials such as hyaluronic acid, carboxymethyl cellulose, chitosan, or algae-based polymers (e.g., alginate, carrageenan), and may further incorporate excipients or delivery-enhancing technologies such as nanoencapsulation, liposomal systems, pH-sensitive coatings, enzyme-responsive carriers, or microcrystalline stabilizers to improve solubility, stability, and targeted release.

The system may be applied as a standalone patch, in combination with wearable devices or functional fabrics, or as part of a multi-phase therapeutic regimen for the management of acute or chronic sciatic nerve symptoms. In certain embodiments, the DMN system may be used as an adjunct to physical therapy, medication, or integrative treatments to accelerate healing, reduce pain, and modulate immune response. While specific examples of therapeutic agents, delivery sites, and use cases are provided, it is understood that the invention includes additional agents and methods not explicitly listed, including as-yet undiscovered compounds or modifications that serve equivalent therapeutic functions.

The present invention further includes a dissolvable microneedle (DMN) system designed for the treatment and prevention of herpes simplex virus type 1 (HSV-1) infections, including cold sores and recurrent oral herpes. The DMNs may be applied to perioral or lip-adjacent regions and deliver one or more antiviral agents directly into the dermal or submucosal layers, enabling rapid and localized suppression of viral replication. The microneedle formulation may include natural, synthetic, or bioengineered antiviral compounds, including but not limited to lysine, zinc salts, glycyrrhizin from licorice root extract, lemon balm extract, monolaurin, or marine-derived agents such as calcium spirulan. These may be combined with anti-inflammatory or wound-healing ingredients such as C-Phycocyanin, aloe vera, or vitamin C to accelerate recovery and reduce tissue damage. The DMNs may be fabricated in dot, oval, or crescent shapes ranging from approximately 3 mm to 15 mm in diameter and may be formulated for immediate, sustained, or staged release. The invention may also include a prophylactic version for individuals with frequent HSV-1 outbreaks, providing localized immune or antiviral support during periods of stress or immune suppression. Algae based compounds used to prevent or treat cold sores would provide a natural and sustainable alternative to synthetic antiviral agents.

The present invention further includes a dissolvable microneedle (DMN) system configured for application to the lips or perioral area for the purpose of enhancing lip hydration, softness, elasticity, and natural volume. The DMN array may be fabricated in curved or heart-shaped patches designed to conform to the upper and lower lips and may deliver one or more bioactive or hydrating agents into the upper dermal layers of the lip tissue. In one embodiment, the DMNs may include algae-based compounds such as Spirulina extract, C-Phycocyanin, astaxanthin, fucoidan, laminarin, or algal peptides, which provide antioxidant, anti-inflammatory, regenerative, or barrier-supportive benefits. These agents may be used alone or in combination with known hydrating and plumping compounds such as hyaluronic acid, glycerin, marine collagen, trehalose, aloe vera, and vitamin B5 (panthenol) to improve moisture retention, reduce chapping, and enhance lip fullness through tissue hydration and natural collagen support. The microneedles may vary in length from approximately 150 μm to 500 μm and may be fabricated using biocompatible polymers such as hyaluronic acid, carboxymethyl cellulose, or alginate. In certain embodiments, the formulation may include encapsulated compounds for time-controlled release or enhanced stability. While specific ingredients and configurations are described herein, the invention encompasses additional combinations, algae-derived compounds, or future-discovered bioactives capable of delivering equivalent or superior cosmetic or therapeutic effects to the lips.

The present invention includes a dissolvable microneedle (DMN) face mask system comprising one or more bioactive compounds derived from algae, marine sources, or related natural or bioengineered sources for the targeted improvement of various skin conditions and enhancement of overall skin health. The DMN face mask may be fabricated in full-face sheets, partial segments, or modular formats, with shapes and dimensions tailored for application to one or more facial regions, including but not limited to the forehead, cheeks, chin, nose, jawline, or periorbital area. The microneedles are composed of biocompatible, biodegradable materials that dissolve upon insertion into the stratum corneum, facilitating the delivery of bioactive compounds into the epidermis and/or dermal layers. In certain embodiments, the formulation may contain algae-based compounds such as Spirulina extract, C-Phycocyanin, astaxanthin, fucoidan, ulvan, laminarin, marine peptides, chlorophyll-containing microalgae, or combinations thereof. These compounds may offer antioxidant, anti-inflammatory, hydrating, regenerative, barrier-supporting, or skin-brightening effects.

In additional embodiments, the microneedles may also include other therapeutic or cosmetic agents, including but not limited to hyaluronic acid, niacinamide, vitamin C, vitamin A derivatives, coenzyme Q10, salicylic acid, green tea extract, marine collagen, zinc, bakuchiol, and botanical compounds. These agents may support sebum regulation, collagen synthesis, pigment modulation, skin hydration, or cellular turnover. The microneedle system may be designed with customizable needle lengths (e.g., approximately 200 μm to 800 μm), microneedle density, and matrix composition suited for various skin types and treatment objectives. The system may optionally include functional materials such as stabilizers, carriers, encapsulation systems (e.g., liposomes, nanoemulsions, polymer-based capsules), or responsive coatings (e.g., pH-, enzyme-, or temperature-sensitive layers) to enhance stability, prolong shelf life, enable controlled release, or target delivery to specific skin environments.

This invention contemplates all suitable combinations, formats, and structural variations of dissolvable microneedle delivery systems intended for dermal or cosmetic application, including configurations designed for specific treatment zones or full-face coverage. It is understood that the invention is not limited to the specific examples described and encompasses all current or future algae-derived, marine-based, synthetic, recombinant, or otherwise bioactive compounds with therapeutic, cosmetic, or preventative relevance. The microneedle mask system may be used for a wide range of skin concerns, including inflammation, oxidative stress, dehydration, dullness, hyperpigmentation, acne, sensitivity, aging, and barrier disruption. The flexible structure and composition of this system support its use across personal care, clinical dermatology, aesthetic medicine, and wellness applications.

The present invention includes a dissolvable microneedle (DMN) system configured for the treatment, prevention, or management of alopecia and related hair loss conditions, such as androgenetic alopecia, alopecia areata, telogen effluvium, stress-induced hair loss, and inflammatory scalp disorders. The DMNs may be applied to the scalp in localized or diffuse regions and are designed to deliver therapeutic agents directly into the dermis, adjacent to the hair follicle matrix, to support follicle stimulation, dermal regeneration, immune modulation, and nutrient delivery. In certain embodiments, the microneedle formulation comprises one or more bioactive compounds derived from algae or marine microorganisms, including but not limited to strains such as Spirulina platensis, Euglena gracilis, Nannochloropsis gaditana, Chlorella vulgaris, Haematococcus pluvialis, Dunaliella salina, Tetraselmis suecica, Ulva lactuca, Laminaria digitata, Codium fragile, and Ecklonia cava.

The algae-derived compounds may include phycobiliproteins, peptides, amino acid complexes, polyunsaturated fatty acids, phlorotannins (such as dieckol and eckol), sulfated polysaccharides (such as fucoidan, ulvan, and laminarin), chlorophyll derivatives, glycolipids, trace minerals, and naturally occurring or bioengineered growth factors. These agents may be selected or combined to promote follicular proliferation, extend the anagen phase, inhibit 5α-reductase activity, reduce oxidative stress, enhance microcirculation, or modulate inflammatory responses associated with hair loss. In certain embodiments, these algae-derived compounds may be used alone or in combination with other supportive bioactives such as hyaluronic acid, marine collagen, panthenol, green tea extract, amino acids, or botanical adaptogens. Encapsulation techniques such as nanoemulsions, liposomal carriers, or polymeric microspheres may be employed to enhance compound stability, enable targeted delivery, or allow for time-controlled release within the follicular region.

The DMNs may be fabricated in various shapes and sizes, including circular patches, elongated strips, or modular scalp-conforming segments designed to treat the crown, hairline, or temporal regions. Microneedle length may range from approximately 200 μm to 1,000 μm, with customized density and matrix formulations based on application site and compound characteristics. The microneedle matrix may be formed from biodegradable, biocompatible polymers such as hyaluronic acid, carboxymethyl cellulose, chitosan, or marine-derived biopolymers including alginate or carrageenan. While specific algae strains, compound classes, and configurations are described herein, the invention broadly encompasses all current and future algae-based, marine-derived, synthetic, or bioengineered compounds that exhibit therapeutic, cosmetic, or preventative effects on the scalp and hair follicle. The system is suitable for both clinical and consumer applications and provides a novel, minimally invasive, and targeted platform for the delivery of regenerative compounds for the management of alopecia and overall scalp health.

The present invention further includes dissolvable microneedle (DMN) systems configured for the delivery of one or more essential or trace minerals for the prevention, treatment, or management of nutrient deficiencies and related health conditions. In certain embodiments, the DMNs are designed as single-phase or multi-layered structures, with each layer containing distinct mineral compounds or mineral combinations, allowing for controlled, sequential, or sustained release profiles. The microneedles may be fabricated in various shapes and sizes and applied transdermally to deliver therapeutic minerals directly into the systemic circulation, bypassing gastrointestinal metabolism and improving bioavailability, for various populations such as those with impaired nutrient absorption, digestive disorders, or swallowing difficulty.

The formulation may include minerals such as iodine, phosphorus, manganese, chromium, molybdenum, boron, vanadium, silicon, selenium, magnesium, copper, zinc, calcium, iron, and fluoride, in any suitable form, such as chelates, salts, oxides, nanoencapsulated forms, or organically bound complexes. In certain embodiments, the minerals may be derived from or inspired by marine sources (e.g., seaweed, algae, coral, marine sediment), terrestrial sources (e.g., rock dust, mineral springs, clay, soil extracts), or biological sources (e.g., fermented plant extracts, fungi, microbial bioaccumulation). The invention further includes all naturally occurring, synthetic, engineered, or as-yet undiscovered mineral forms or mineral complexes that may deliver equivalent or superior nutritional, therapeutic, or preventative effects.

In some embodiments, the DMN system may include excipients, carriers, stabilizers, or encapsulation technologies such as solid lipid nanoparticles, liposomes, polymeric microspheres, nanoemulsions, or ion-exchange delivery matrices to enhance dermal penetration, prolong shelf life, or enable pH-or enzyme-triggered release. The microneedle matrix may be constructed using biodegradable polymers such as hyaluronic acid, alginate, carboxymethyl cellulose, chitosan, or marine-derived polysaccharides. The system may be used for general wellness, immune support, metabolic regulation, thyroid function, cognitive support, bone health, skin and hair maintenance, or other health outcomes associated with mineral status. While specific mineral examples and delivery methods are provided herein, the invention encompasses all mineral compositions and transdermal delivery technologies suitable for use in a dissolvable microneedle format, now known or hereafter discovered.

The present invention further includes dissolvable microneedle (DMN) systems configured for veterinary applications, including but not limited to the treatment, prevention, or management of disease, nutritional deficiency, reproductive function, stress, parasitic infestation, or post-operative recovery in animals. In certain embodiments, the DMNs may be applied to the skin, gumline, or other transdermally accessible sites in domestic animals, livestock, wildlife, or exotic species. The system is designed to enable painless, stress-free delivery of one or more active agents via dissolvable microneedles composed of biocompatible, biodegradable polymers such as hyaluronic acid, alginate, or chitosan.

The active agents may include nutrients (e.g., vitamin B12, selenium, zinc), vaccines, hormones, anti-parasitic agents (e.g., ivermectin, neem extract), calming compounds (e.g., L-theanine, melatonin), immune modulators, or algae-derived therapeutics such as C-Phycocyanin, Spirulina extract, or fucoidan. The microneedles may vary in shape, size, and needle length (e.g., 200 μm to 1,000 μm) and may be fabricated as strips, dots, patches, or animal-specific conforming designs. In certain embodiments, the DMN patches may be designed for field deployment, self-application through environmental interaction (e.g., rubbing posts), or species-specific targeting, and may incorporate stabilizers or encapsulation systems to enhance durability and storage life in diverse climates. While specific formulations and use cases are provided, the invention includes all current and future veterinary-relevant compounds, delivery methods, and animal-specific configurations suitable for integration into a dissolvable microneedle platform. The use of DMNs for non-invasive veterinary delivery of bioactives, including those for behavioral management, nutrient supplementation, oral care, and field-based health interventions, can provide alternative options to oral and injectable therapies.

The present invention encompasses dissolvable microneedle (DMN) systems for the delivery of one or more fertility-modulating agents to animals, such as to companion animals, livestock, captive species, and free-ranging wildlife. The DMNs are configured to administer biologically active compounds via the skin or mucosal tissue using minimally invasive, biodegradable microneedle arrays that dissolve upon application, enabling localized or systemic delivery. These systems may be used for the temporary or extended suppression of reproductive function, estrus regulation, population management, or contraception, and may serve as an alternative to conventional delivery methods such as injection, implants, oral dosing, or surgical sterilization.

In various embodiments, the DMN formulation may comprise one or more compounds capable of modulating reproductive pathways, such as hormonal contraceptives, immunocontraceptive agents, reproductive hormone analogs or antagonists, and other naturally occurring, synthetic, or bioengineered fertility regulators. By way of example only and without limitation, such agents may include synthetic or natural progestogens, gonadotropin-releasing hormone (GnRH) agonists or antagonists, luteinizing hormone (LH) modulators, peptide-based vaccines targeting reproductive hormones or receptors, or biologically derived antigens such as zona pellucida (ZP) glycoproteins. In certain embodiments, compounds such as medroxyprogesterone acetate (MPA), melengestrol acetate (MGA), altrenogest, progesterone, deslorelin, or other structurally or functionally similar molecules may be utilized. These may be delivered as free agents or in encapsulated, complexed, or stabilized forms to allow for time-controlled, staged, or extended release.

The microneedles may be fabricated using biocompatible, biodegradable materials suitable for veterinary use, such as hyaluronic acid, chitosan, alginate, gelatin, cellulose derivatives, marine-derived polysaccharides, or other pharmaceutically acceptable polymers. The DMNs may be produced in a variety of forms, including transdermal patches, strips, or spot applicators, and may be configured for application to anatomical regions with minimal hair coverage, such as behind the ear, along the flank, inner thigh, or other accessible locations depending on the species. Microneedle length, density, and composition may vary to accommodate species-specific dermal thickness, intended duration of release, or desired depth of delivery.

While specific agents, formats, and applications are described herein, the present invention is not limited to the embodiments provided, and encompasses all compounds, delivery strategies, structural variations, and use cases suitable for controlling animal fertility through a dissolvable microneedle-based platform. This includes compounds and technologies now known or hereafter discovered that are functionally equivalent or provide similar outcomes. The invention is applicable across clinical, field-based, and mobile deployment settings, including for veterinary, livestock, and wildlife management applications where minimally invasive, sustained, or field-friendly contraceptive delivery is beneficial.

The present invention relates to dissolvable microneedle (DMN) systems formulated to support the remineralization of teeth, strengthen oral tissues, and enhance oral wellness in both human and veterinary applications. These systems are configured for application to the oral cavity or adjacent external areas such as the gumline, inner cheeks, or perioral skin, allowing for localized or systemic delivery of bioactive compounds through transdermal or transmucosal pathways. The DMNs may be used for the enhancement of enamel integrity, dentin support, reduction of tooth sensitivity, or overall maintenance of oral tissue health.

The microneedle formulation may include one or more compounds capable of supporting mineralization, biological repair, or functional regeneration of oral structures. These compounds may include macro or trace minerals, mineral cofactors, or bioactive agents of natural or engineered origin. Examples of suitable compounds include, without limitation, biologically compatible forms of calcium, magnesium, phosphorus, silica, boron, and zinc, as well as rare trace minerals found in marine sediments or mineral-rich environments. In certain embodiments, the mineral source may be derived from algae, seaweed, geothermal deposits, soil microorganisms, fermented biomass, or bioaccumulated plant complexes. These may be used alone or in combination with biologically active peptides, amino acids, or natural compounds that support oral tissue hydration, collagen synthesis, antioxidant defense, or microbial balance.

The formulation may optionally include structurally or functionally supportive materials, such as polysaccharide-based carriers, mineral-binding proteins, or stabilizing biogels. The microneedles may be produced in various shapes, lengths, and densities depending on anatomical site and species-specific design considerations. Configurations may include patches or strips for application to the gumline, inner cheek, roof of the mouth, or tooth-adjacent areas, and may be customized for use in animals with unique oral anatomies. The microneedles may range in length from approximately 200 to 800 micrometers and be composed of biodegradable, biocompatible materials suitable for use in the oral cavity, including natural polymers and marine-derived gels.

This invention encompasses all known and future-discovered minerals, mineral complexes, bioactive compounds, and delivery system designs capable of enhancing oral mineral content or promoting tissue integrity through the use of dissolvable microneedle-based systems, whether for preventive, cosmetic, therapeutic, or wellness applications. No limitation is placed on the specific mineral forms, sources, or combinations beyond their functional compatibility with the microneedle delivery platform and their intended benefit to oral or dental health.

The present invention relates to dissolvable microneedle (DMN) systems designed for the prevention, treatment, and management of oral abscesses and associated bacterial infections in both human and veterinary applications. The DMNs are configured to deliver antimicrobial agents directly to oral tissues, such as the gingiva, inner cheek, jawline, or mucosal surfaces, allowing for targeted, site-specific delivery while minimizing systemic exposure and bypassing gastrointestinal metabolism.

In certain embodiments, the DMN formulation may include one or more antibacterial or antimicrobial agents that have not previously been incorporated into microneedle systems for the treatment of oral abscesses. These agents may be synthetic, semi-synthetic, natural, or marine-derived, and may be used individually or in combination. Synthetic antibiotics that may include clindamycin, amoxicillin, metronidazole, doxycycline, cephalexin, azithromycin, rifampin, linezolid, fosfomycin, ornidazole, and tinidazole, as well as veterinary-appropriate agents such as enrofloxacin, marbofloxacin, and tylosin. These may be delivered in stabilized or encapsulated form using polymers, liposomes, microspheres, or nanocarriers to enhance delivery, solubility, and bioavailability.

The invention also includes natural and marine-based antimicrobials, which may be used alone or synergistically with synthetic antibiotics. Examples include allicin (from garlic), berberine (from Berberis), propolis extract, curcumin, eugenol (from clove), thymol, and other essential oil components, as well as marine-derived agents such as phlorotannins from Ecklonia cava, sulfated polysaccharides like fucoidan and ulvan, bromophenols from red algae, sea sponge derived antimicrobial peptides, and marine bacterial lipopeptides. These compounds may provide broad-spectrum or selective antibacterial effects while also offering anti-inflammatory or wound-healing support.

The microneedles may be fabricated in various formats such as gumline strips, intraoral patches, buccal inserts, circular spot patches, or transdermal applications along the jawline or facial region. Microneedle height may range from approximately 200 to 800 micrometers, with matrix materials such as chitosan, marine-derived polysaccharides, alginate, gelatin, or hyaluronic acid. Formulations may be designed for rapid or sustained release, and may include co-ingredients that promote tissue regeneration, reduce pain, or modulate immune response.

While specific compounds and formats are provided for illustrative purposes, the invention broadly encompasses all natural, synthetic, marine-based, or bioengineered antimicrobial agents, including those not yet discovered, that are compatible with delivery through dissolvable microneedles for the treatment or prevention of oral abscesses or related infections. The system may be used across clinical, home care, or veterinary settings and may be combined with other therapeutic agents for comprehensive oral infection management or oral health.

The present invention includes dissolvable microneedle (DMN) systems formulated to disrupt, prevent, and manage dental plaque and associated oral biofilms in both humans and animals. These systems are configured to deliver antibacterial, anti-biofilm, or enzymatic agents to the gingiva, oral mucosa, or tooth-adjacent regions via localized transdermal or transmucosal delivery. The DMNs may be used as a standalone oral hygiene solution, as an adjunct to mechanical cleaning, or in cases where brushing and rinsing are ineffective or impractical, such as for pediatric, geriatric, or veterinary populations.

In certain embodiments, the formulation includes compounds capable of disrupting biofilm architecture, inhibiting bacterial adhesion, or neutralizing bacterial byproducts. Such compounds may include enzymes (e.g., dextranase, mutanase, lysozyme, or proteases), natural antibiofilm agents (e.g., zinc citrate, copper peptides, chitosan, propolis, catechins from green tea, or polyphenols from pomegranate), marine-derived antimicrobials (e.g., fucoidan, phlorotannins, sulfated polysaccharides, bromophenols), or synthetic agents (e.g., chlorhexidine alternatives, bisbiguanides, or anti-quorum sensing molecules). These may be combined with stabilizers, hydrating agents, or adjunct compounds such as arginine, xylitol, or calcium phosphate complexes to support remineralization and oral tissue recovery.

The DMNs may be fabricated in various forms such as gumline-adhering strips, buccal film patches, or spot-applicable dot patches for areas of plaque buildup. The microneedles may range from 200 to 800 micrometers in length and be composed of biodegradable, biocompatible polymers suitable for oral use, such as hyaluronic acid, alginate, chitosan, or marine gel matrices. The formulation may optionally include agents for sustained release, pH-sensitive dissolution, or enzyme-triggered activation in response to the acidic biofilm environment. While specific examples are described, the invention encompasses all naturally occurring, synthetic, marine-derived, or bioengineered compounds capable of reducing, disrupting, or inhibiting plaque formation or bacterial colonization using a dissolvable microneedle-based delivery platform.

It is to be understood that many other uses for a DMN patch exist beyond the ones described above, and that the invention is not limited by these particular uses.

In certain embodiments, the active pharmaceutical ingredient, biologic, or bioactive compound delivered via dissolvable microneedles may exhibit a range of release and retention durations in the body, depending on the formulation, encapsulation method, and delivery strategy employed. In some cases, the compound may be released immediately upon microneedle dissolution, with systemic absorption occurring within minutes to a few hours; this is suitable for vaccines, peptides, or fast-acting small molecules. In other embodiments, short-acting release may be achieved, with drug presence maintained for approximately 6 to 24 hours through modulation of polymer composition or microneedle thickness. Intermediate-release profiles may be realized through the inclusion of biodegradable nanoparticles, liposomes, or microspheres within the microneedle matrix, enabling sustained release over a period of several days to several weeks. In further embodiments, long-acting release may be achieved for durations extending up to 365 days. This may be accomplished through microneedles that implant a depot-forming system—such as poly(lactic-co-glycolic acid) (PLGA) microspheres, hybrid lipid-polymer nanoparticles, or thermosensitive gel implants into the dermal or subdermal layer. Upon dissolution of the microneedles, the implanted material degrades or releases its payload gradually over time, mimicking the performance of injectable long-acting depot formulations. This approach enables minimally invasive, extended drug delivery while avoiding repeated administration. The specific release duration can be tailored by adjusting the carrier system's degradation rate, particle size, polymer molecular weight, or crosslinking density. All such release durations from immediate to ultra-long-acting are considered within the scope of this invention, including systems designed to achieve therapeutic effects lasting three months or longer through bioresponsive or depot-forming microneedle delivery platforms.

In certain embodiments, the microneedle system may comprise bioactive compounds encapsulated within depot-forming carriers, such as biodegradable polymers (e.g., poly (lactic-co-glycolic acid) (PLGA), polycaprolactone), which may remain at the application site and release the bioactive over an extended period. These depot systems may enable controlled or sustained release ranging from several days to several months, or up to one year. Such long-acting delivery is suitable for chronic disease therapies, contraceptives, hormone replacement, long-term nutrient supplementation, or seasonal vaccinations. By integrating nanoparticle or microsphere-based formulations into the microneedle matrix, and optionally modulating the polymer degradation rate, the system can achieve pharmacokinetically relevant plasma levels for extended durations after a single administration.

1. A dissolvable microneedle system, comprising:
a. an array of dissolvable microneedles, each microneedle comprising a biocompatible material capable of dissolving in a living being's interstitial fluid;
b. at least one bioactive compound embedded in each of the array of dissolvable microneedles, wherein the bioactive compound comprises one or more of: proteins, peptides, amino acids, antioxidants, polysaccharides, polyphenols, pigments, fatty acids, minerals, vitamins, enzymes, synthetic small molecules, botanical extracts, plant-derived volatile compounds, and naturally derived phytochemical compounds;
c. wherein each of the dissolvable microneedles is capable of penetrating a living being's skin.

2. The dissolvable microneedle system of claim 1, wherein the bioactive compound is encapsulated in at least one of solid lipid nanoparticles, nanostructure lipid carriers, polymeric nanoparticles, dendrimers, liposomes, micelles, hybrid nanoparticles, and cyclodextrin complexes, niosomes, ethosomes, cubosomes, nanocrystals, protein-based nanoparticles, mesoporous silica nanoparticles, exosomes, and biopolymer-based carriers.

3. The dissolvable microneedle system of claim 2, wherein the bioactive compound is encapsulated in solid lipid nanoparticles.

4. The dissolvable microneedle system of claim 3, wherein the bioactive compound is at least one of the following: compound derived from algae, whole Spirulina, C-Phycocyanin.

5. The dissolvable microneedle system of claim 1, wherein the bioactive compound has a molecule size larger than 4 kilodaltons.

6. The dissolvable microneedle system of claim 3, wherein the bioactive compound is at least one amino acid or peptide derived from algae.

7. The dissolvable microneedle system of claim 1, wherein the bioactive compound is a stabilized biologic, wherein the biologic is a macromolecule selected from proteins, peptides, antibodies, antibody fragments, nucleic acids, aptamers, cytokines, receptors, fusion proteins, or analogues thereof, and wherein the biologic is encapsulated in a carrier and wherein the system is configured for topical, transdermal, or mucosal delivery.

8. The dissolvable microneedle system of claim 7, wherein the carrier is selected from solid lipid nanoparticles, polymeric nanoparticles, liposomes, nanoemulsions, microspheres, nanostructured lipid carriers, hydrogel nanoparticles, dendrimers, exosome mimicking vesicles, or depot-forming biodegradable matrices, and wherein the carrier is configured to enable controlled release of the biologic over an extended duration.

9. The dissolvable microneedle system of claim 7, wherein the biologic is co-formulated with a stabilizer selected from trehalose, mannitol, polyethylene glycol (PEG), or surfactants selected to preserve the conformation and bioactivity of the biologic.

10. The dissolvable microneedle system of claim 1, wherein each microneedle has a pyramidal, conical, barbed, asymmetric, multilayered, or hybrid geometry.

11. The dissolvable microneedle system of claim 1, wherein each microneedle has a height to base ratio between 1.5:1 and 5:1.

12. The dissolvable microneedle system of claim 1, wherein each microneedle has a height ranging from 100 μm to 2000 μm.

13. The dissolvable microneedle system of claim 1, wherein each microneedle has a base ranging from 100 μm to 500 μm in diameter.

14. The dissolvable microneedle system of claim 1, wherein a spacing between neighboring microneedles ranges from 300 μm to 1200 μm.

15. The dissolvable microneedle system of claim 1, wherein each microneedle has a minimum fracture force that is greater than 0.058 N and an insertion force ranging from 0.1 N to 3.0 N.

16. The dissolvable microneedle system of claim 1, wherein the biocompatible material comprises at least one of hyaluronic acid, pullulan, chitosan, or alginate.

17. The dissolvable microneedle system of claim 16, further comprising crosslinked forms created by a crosslinking agent, wherein the crosslinking agent is a natural or plant-derived compound selected from the group consisting of genipin, citric acid, tannic acid, or other biocompatible crosslinkers.

18. The dissolvable microneedle system of claim 1, wherein each microneedle further comprises an excipient, wherein the excipient is selected from a group comprising stabilizers, plasticizers, solubilizers, dispersants, disintegrants, humectants, antioxidants, and buffering agents.

19. The dissolvable microneedle system of claim 1, wherein each microneedle further comprises a nanoparticle-stabilizing agent, wherein the nanoparticle-stabilizing agent is selected from a group comprising poloxamers, polysorbates, lecithin, saponins, or plant-derived surfactants.

20. The dissolvable microneedle system of claim 1, wherein each microneedle comprises at least two parts, a first part and a second part, wherein the first part comprises a first biocompatible material and the second part comprises a second biocompatible material, wherein the first biocompatible material dissolves more quickly than the second biocompatible material.

21. The dissolvable microneedle system of claim 1, wherein the microneedles are configured for immediate, biphasic, sustained, or controlled release over a period ranging from a few seconds to 365 days.

22. The dissolvable microneedle system of claim 1, wherein the system is configured as one of the following: a face mask, spot patch, gumline strip, full-body wrap, under-eye crescent, lip patch, oral insert, transdermal wearable, veterinary patch, textile item, or agricultural film for application to plant surfaces.

23. The dissolvable microneedle system of claim 1, further comprising an embedded indicator configured to provide a detectable signal corresponding to the degree of microneedle dissolution or release of the bioactive compound wherein the embedded indicator comprises at least one of the following:
a. a colorimetric dye or pigment that changes color upon microneedle dissolution or release of the bioactive compound;
b. an element that emits a fluorescent or optically detectable signal configured to be detected by a camera, wearable scanner, mobile device, or software interface;
c. an element that is chemically responsive to one or more environmental conditions selected from hydration, pH, enzymatic activity, friction, interstitial fluid composition, body temperature, or electrical resistance.

24. A method of treatment, prevention, or management of a medical condition, comprising:
a. creating a DMN comprising a bioactive compound applicable to the medical condition;
b. applying the DMN to a living being for the treatment, prevention, or management of the condition.

25. The method of claim 23, wherein the condition is one of the following: neurological, psychological.

26. The method of claim 23, wherein the condition is one of the following: a digestive disorder, a nutritional deficiency.

27. The method of claim 23, wherein the condition is autoimmune.

28. The method of claim 23, wherein the condition is one of the following: post-viral, infectious.

29. The method of claim 23, wherein the bioactive compound is configured to modulate inflammation, oxidative stress, sleep, athletic performance, immune function, neurological activity, hormonal balance, or metabolic processes.

30. The method of claim 23, wherein the bioactive compound is configured to support hair growth, manage scalp inflammation, manage sebum balance, manage the skin microbiome, support skin regeneration, hydration, wound healing, melanin, skin aging, or collagen production.
